(19) United States
(12) Patent Application Publication
(10) Pub. No.: US 2011/0207751 A1
(43) Pub. Date: Aug. 25, 2011

(54) HETEROCYCLIC COMPOUNDS AND USES AS ANTICANCER AGENTS

(76) Inventors: Long Mao, San Diego, CA (US); Xiaobo Wang, San Diego, CA (US); Xiao Xu, San Diego, CA (US); Li Zhao, San Diego, CA (US); Jia Liu, San Diego, CA (US)

(21) Appl. No.: 13/031,134

(22) Filed: Feb. 18, 2011

Related U.S. Application Data
(60) Provisional application No. 61/306,416, filed on Feb. 19, 2010.
(60) Provisional application No. 61/314,510, filed on Mar. 16, 2010.

(51) Int. Cl. A61K 31/519; C07D 487/04; A61P 35/00; A61P 35/02; A61K 31/429; C07D 513/04; A61K 31/4439

(52) U.S. Cl. 514/259.1; 544/281; 514/368; 548/154; 514/338; 546/270.1

(57) ABSTRACT

Novel compounds having a fused bicyclic heteroaromatic ring system substituted with a thiazole ring are disclosed. The compounds inhibit growth of a variety of types of cancer cells, and are thus useful for treating cancer. Efficacy of these compounds is demonstrated with a system for monitoring cell growth/migration, which shows they are potent inhibitors of growth and/or migration of cancer cells. In addition, compounds of the invention were shown to stop growth of tumors in vivo, and to reduce the size of tumors in vivo. Compositions comprising these compounds, and methods to use these compounds and compositions for treatment of cancers, are disclosed.

### CROSS-REFERENCE TO RELATED APPLICATIONS

This application claims priority to U.S. Provisional Patent Application Nos. 61/306,416, filed Feb. 19, 2010 and 61/314,510 filed Mar. 16, 2010, the disclosures of which are hereby incorporated herein by reference in their entireties.

### FIELD OF THE INVENTION

The field of the invention is heterocyclic compounds, pharmaceutical compositions and methods, and especially as they relate to compositions and methods for the treatment and prevention of cancer and related diseases.

### BACKGROUND OF THE INVENTION

Cancer is the second biggest cause of death in the developed countries. Therefore, cancer remains one of the most important unmet medical challenges to mankind A number of options for treating tumors are available, including surgery, radiation, chemotherapy, or any combination of these approaches. Among these, chemotherapy is widely used for all types of cancers, in particular for those inoperable or with metastatic characteristics. Despite a variety of chemotherapeutic compounds being used in clinics for improvement of survival rates of different human cancers, chemotherapy is generally not curative, but only delays disease progression.

Commonly, tumors and their metastasis become refractory to chemotherapy, as the tumor cells develop the ability of multi-drug resistance. In some cases, the tumors are inherently resistant to some classes of chemotherapeutic agents. In other cases, the acquired resistance against chemotherapeutic agents is developed during the chemotherapeutic intervention. Thus, there remain significant limitations to the efficacy of available chemotherapeutic compounds in treating different classes of tumors. Furthermore, many cytotoxic and cytostatic agents used for chemotherapeutic treatment of tumors have severe side effects, resulting in termination of the chemotherapy in some patients. Thus, there remains a need for new chemotherapeutic agents to treat cancer.

### BRIEF SUMMARY OF INVENTIONS

The present invention is directed to novel compounds having a bicyclic heteroaryl ring system linked to an aniline-substituted thiazole ring, pharmaceutical compositions containing these compounds, and methods of using these compounds and compositions. The compounds as described herein exhibit anti-tumor, anticancer, anti-inflammation, anti-infectious, and antiproliferation activity. They are particularly useful for treatment of cancers as demonstrated by selective toxicity to a cancer cells, including many different types of cancers. The present invention also relates to pharmaceutical compositions containing such compounds, which may be used to treat tumors, cancer, and infective and/or proliferative diseases.

In one aspect of the inventive subject matter, the novel heterocyclic compounds have a structure according to Formula I or II:

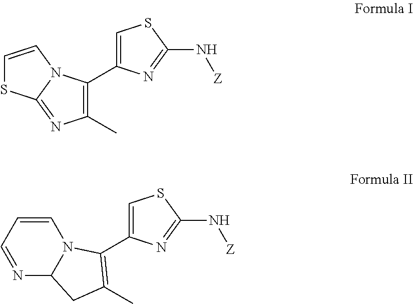

where Z is selected but not limited from the following substituted phenyl or heterocyclic rings (the bond bisected by a dashed line in these structures represents the point of attachment of the Z group to NH in formula I or II):

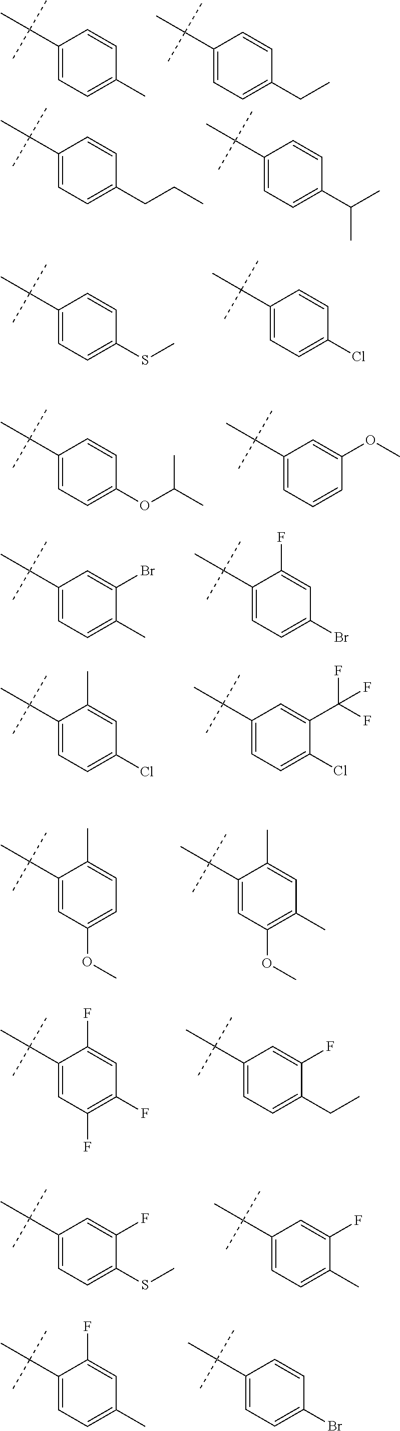

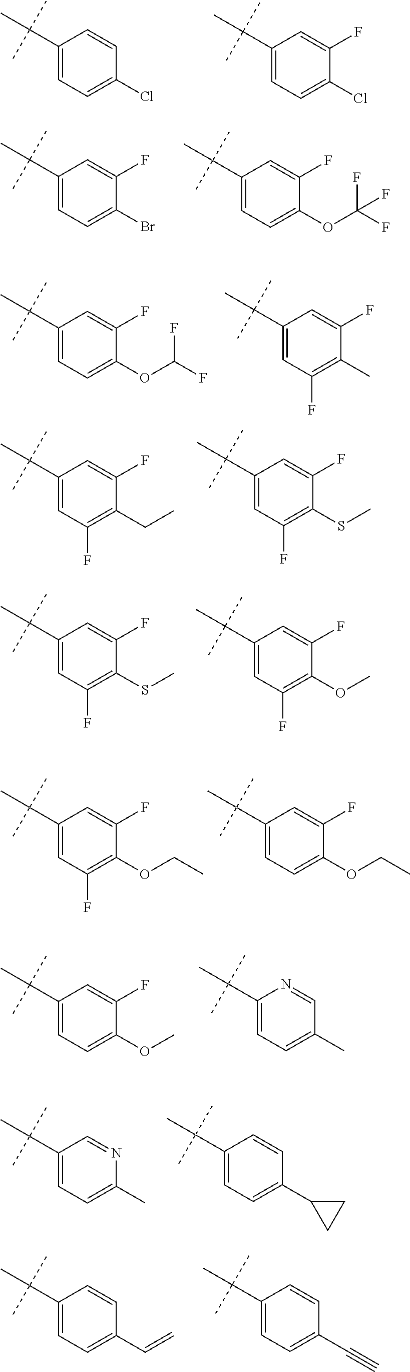

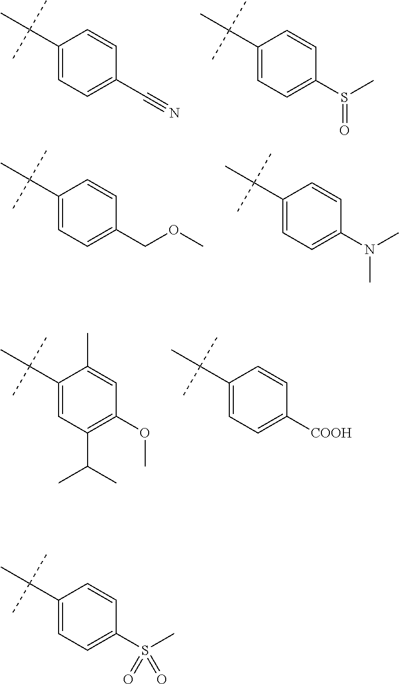

or a pharmaceutically acceptable salt or acylated prodrug thereof.

Compounds similar to those described herein have been reported (WO 2009/023402), including use of such compounds for treating cancer. However, the novel compounds described herein are unexpectedly superior to compounds known in the art.

The compounds of formulas I-II can be used as neutral compounds or as their pharmaceutically suitable salts with inorganic and organic counterions. Their salts include acid addition salts comprising a pharmaceutically acceptable counterion, such as, but not limited to, halides (Cl⁻, Br⁻, I⁻), nitrate, mesylate, p-toluene sulfonate/tosylate, oxalate, citrate, malate, maleate, tartrate, fumarate, formate, acetate and similar anions in these classes.

The above-described heterocyclic compounds include the compounds themselves, as well as their salts and their prodrugs, if applicable. Such salts, for example, can be formed between a positively charged substituent group (e.g., an amino group on heterocyclic or aromatic rings that is protonated) on a compound and a pharmaceutically suitable anion, or by addition of an acid to a basic heterocyclic group of the compounds of Formulas I or II. Suitable anions include, but are not limited to, chloride, bromide, iodide, sulfate, nitrate, phosphate, citrate, benzenesulfonate, methanesulfonate, trifluoroacetate, maleate, and acetate. Similarly, a negatively charged substituent (e.g., carboxylate group on heterocyclic or aromatic rings) on a compound can form a salt with a pharmaceutically-acceptable cation. Non-limiting examples of suitable cations are sodium ion, potassium ion, magnesium ion, calcium ion, and a organic ammonium ion such as teteramethylammonium ion, tetrabutylammonium ion, and other organic cations.

Suitable prodrugs can be formed by acylation of the NH group of Formula I or II. Exemplary prodrugs include compounds of Formula I or II wherein NH has been acylated to NC(O)—R*, where C(O)R* is an optionally substituted acyl group such as formyl, acetyl, chloroacetyl, trichloroacetyl, trifluoroacetyl, and the like. Other prodrugs include compounds of Formula I where the NH has been sulfonylated, to form e.g. N—SO₂—R′, where R′ can be methyl, fluoromethanesulfonyl, or trifluormethanesulfonyl, for example.

Compounds of the invention may exist as isomers, including optical isomers, geometric isomers, tautomers, and rotational isomers including atropisomers. The invention includes each such isomer of the compounds of formula I-II, and mixtures thereof. Where a compound has a chiral center, for example, the invention includes each individual isomer as well as mixtures of both isomers in varying amounts, including a racemic mixture having equal amounts of both isomers. Because the compounds of the invention are biaryls, they can exist as rotational isomers about the biaryl linkage, also, and each isomer as well as mixtures of such isomers are included within the scope of the invention.

The compounds and compositions comprising the compounds of the invention are useful to treat conditions characterized by undesired cell proliferation. In particular, the compounds are useful to treat sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head and neck cancers, pancreatic cancer, and other types of proliferative diseases.

### BRIEF DESCRIPTION OF THE DRAWINGS

FIG. 1 shows the in vivo antitumor efficacy of COMPOUND O on MKN45 human Gastric-intestinal Cancer that was xenograft-transplanted in immunodeficient nude mice by subcutaneous implanting.

FIG. 2 shows the in vivo antitumor efficacy of COMPOUND O on H460 human Non-small cell lung Cancer that was xenograft-transplanted in immunodeficient nude mice by subcutaneous implanting.

FIG. 3 shows the in vivo antitumor efficacy of COMPOUND O on A549 human Non-small cell lung Cancer that was xenograft-transplanted in immunodeficient nude mice by subcutaneous implanting.

FIG. 4 shows the dynamic response profiles of A549 human non-small cell lung carcinoma cell line to different concentrations of COMPOUND O (FIG. 4A), paclitaxel (FIG. 4B) and vincristine (FIG. 4C) as determined on Real-Time Cell Electronic Sensing (RT-CES system from ACEA Biosciences, which is the same as xCELLigence system from Roche).

FIG. 5 shows the dynamic response profiles of H596 human adenosquamous lung carcinoma cell line to different concentrations of COMPOUND O as determined on xCelligence system (Roche), which is the same as Real-Time Cell Electronic Sensing (RT-CES) system (ACEA Biosciences).

FIG. 6 shows the dynamic response profiles of H292 human pulmonary lung carcinoma cell line to different concentrations of COMPOUND O as determined on xCelligence system (Roche).

FIG. 7 shows the dynamic response profiles of H460 human large cell lung carcinoma cell line to different concentrations of COMPOUND O as determined on xCelligence system (Roche).

FIG. 8 shows the dynamic response profiles of H1993 human non-small cell lung cancer cell line to different concentrations of COMPOUND O as determined on xCelligence system (Roche).

FIG. 9 shows the dynamic response profiles of H1838 human non-small cell lung cancer cell line to different concentrations of COMPOUND O as determined on xCelligence system (Roche).

FIG. 10 shows the dynamic response profiles of H2347 human non-small cell lung cancer cell line to different concentrations of COMPOUND O as determined on xCelligence system (Roche).

FIG. 11 shows the dynamic response profiles of SW620 human colon cancer cell line to different concentrations of COMPOUND O as determined on xCelligence system (Roche).

FIG. 12 shows the dynamic response profiles of GTL16 human gastric cancer cell line (which was derived from MKN45 human gastric cancer cell line) to different concentrations of COMPOUND O as determined on xCelligence system (Roche).

FIG. 13 shows the dynamic response profiles of HT29 human colon cancer cell line to different concentrations of COMPOUND O as determined on xCelligence system (Roche).

FIG. 14 shows the dynamic response profiles of A172 human brain cancer cell line to different concentrations of COMPOUND O as determined on xCelligence system (Roche).

FIG. 15 shows the dynamic response profiles of U138MG human brain cancer cell line to different concentrations of COMPOUND O as determined on xCelligence system (Roche).

FIG. 16 shows the dynamic response profiles of U118MG human brain cancer cell line to different concentrations of COMPOUND O as determined on xCelligence system (Roche).

FIG. 17 shows the dynamic response profiles of SW1088 human brain cancer cell line to different concentrations of COMPOUND O as determined on xCelligence system (Roche).

FIG. 18 shows the dynamic response profiles of HT1080 human connective tissue cancer cell line to different concentrations of COMPOUND O as determined on xCelligence system (Roche).

FIG. 19 Shows the dynamic response profiles of B×PC3 human pancreatic cancer cell line to different concentrations of COMPOUND O as determined on xCelligence system (Roche).

FIG. 20 shows the dynamic response profiles of HepG2 human liver cancer cell line to different concentrations of COMPOUND O as determined on xCelligence system (Roche).

FIG. 21 Shows the dynamic response profiles of SKOV3 human ovarian cancer cell line to different concentrations of COMPOUND O as determined on xCelligence system (Roche).

FIG. 22 shows the dynamic response profiles of MCF7 human breast cancer cell line to different concentrations of COMPOUND O as determined on xCelligence system (Roche).

FIG. 23 shows the dynamic response profiles of MDA-MB-231 human breast cancer cell line to different concentrations of COMPOUND O as determined on xCelligence system (Roche).

FIG. 24 shows the dynamic response profiles of KB human cervical cancer cell line (FIG. 24A-D) and of KB200 human cervical cancer cell lines (expressing multiple drug resistant MDR gene) (FIG. 24E-H) to different concentrations of COMPOUND O (FIGS. 24A and E), paclitaxel (FIGS. 24B and F), vinblastine (FIGS. 24C and G) and colchicine (FIGS. 24D and H) as determined on xCelligence system (Roche).

FIG. 25 shows the dynamic response profiles of NIH3T3 the normal tissue cell line to different concentrations of COMPOUND O as determined on xCelligence system (Roche).

FIG. 26A shows the effect of COMPOUND O on microtubule assembly in vitro using MAP-rich tubulin.

FIG. 26B shows the inhibition effect of microtubule organization in A549 cells by 20 hr treatment of COMPOUND O, paclitaxel and vincristine.

FIG. 26C shows the interaction of COMPOUND O with tubulin via a colchicine-binding site using a spin column assay.

FIG. 27A shows the apoptosis of A549 human lung cancer cells, as induced by 24 hr, 48 hr and 72 hr treatment of 37 nM COMPOUND O and 37 nM paclitaxel.

FIG. 27B shows the apoptosis of A549, H596 and H292 human lung cancer cells, as induced by 72 hr treatment of 37 nM COMPOUND O and 37 nM paclitaxel.

FIG. 28A shows the extent of mitotic arrest of A549 human lung cancer cells by paclitaxel and COMPOUND O, as quantified by the mitotic index.

FIG. 28B shows the cell cycle distribution of A549 human lung cancer cells after 24 hr treatment with COMPOUND O.

### EMBODIMENTS OF THE INVENTION

For clarity of disclosure, and not by way of limitation, the following description of selected embodiments of the invention is provided. It is divided into the subsections that follow for convenience and the section divisions should not be read to limit the scope of the invention.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition incorporated herein by reference.

As used herein, “a” or “an” means “at least one” or “one or more”.

The term “alkyl” as used herein refers to saturated hydrocarbon groups in a straight, branched, or cyclic configuration and particularly contemplated alkyl groups include lower alkyl groups (i.e., those having ten or less carbon atoms). Exemplary alkyl groups are methyl, ethyl, propyl, isopropyl, butyl, sec-butyl, tertiary butyl, pentyl, isopentyl, hexyl, etc. The term “alkenyl” as used herein refers to an alkyl as defined above and having at least one double bond. Thus, particularly contemplated alkenyl groups include straight, branched, or cyclic alkenyl groups having two to ten carbon atoms (e.g., ethenyl, propenyl, butenyl, pentenyl, etc.). Similarly, the term “alkynyl” as used herein refers to an alkyl or alkenyl as defined above and having at least one triple bond. Especially contemplated alkynyls include straight, branched, or cyclic alkynes having two to ten total carbon atoms (e.g., ethynyl, propynyl, butynyl, etc.).

The term “cycloalkyl” as used herein refers to a cyclic alkane (i.e., in which a chain of carbon atoms of a hydrocarbon forms a ring), preferably including three to eight carbon atoms. Thus, exemplary cycloalkanes include cyclopropyl, cyclobutyl, cyclopentyl, cyclohexyl, cycloheptyl, and cyclooctyl. Cycloalkyls also include one or two double bonds, which form the “cycloalkenyl” groups. Cycloalkyl groups are also further substituted by alkyl, alkenyl, alkynyl, halo and other general groups.

The term “aryl” or “aromatic moiety” as used herein refers to an aromatic ring system, which may further include one or more non-carbon atoms. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group, and are thus termed “fused aryl” or “fused aromatic”.

As also used herein, the terms “heterocycle”, “cycloheteroalkyl”, and “heterocyclic moieties” are used interchangeably herein and refer to any compound in which a plurality of atoms form a ring via a plurality of covalent bonds, wherein the ring includes at least one atom other than a carbon atom. Particularly contemplated heterocyclic bases include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Further contemplated heterocycles may be fused (i.e., covalently bound with two atoms on the first heterocyclic ring) to one or two ring or heterocycle, and are thus termed “fused heterocycle” or “fused heterocyclic base” or “fused heterocyclic moieties” as used herein.

The term “halogen” as used herein refers to fluorine, chlorine, bromine and iodine.

It should further be recognized that all of the above-defined groups may further be substituted with one or more substituents, which may in turn be substituted as well. For example, a hydrogen atom in an alkyl or aryl is substituted with an amino, halo or other groups.

The term “substituted” as used herein refers to a replacement of an H atom with another atom or group. Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

Particularly contemplated functional groups include nucleophilic groups (e.g., —NH₂, —OH, —SH, —NC, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃⁺), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, PO₃H, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties. Further more, the term “mono-/di-/tri-/tetra-substituted” used herein refers to one, or two, or three or four functional groups described above that substituted onto the aromatic or heterocyclic or fused aromatic or heterocyclic moiety, in which such multi-functional groups are substituted at the combination of any ortho- or para- or meta-position of the aromatic or heterocyclic moiety.

Additionally, any formula given herein is intended to represent hydrates, solvates and polymorphs of such compounds, and mixtures thereof.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration. “Pharmaceutically acceptable salts” are those salts which retain at least some of the biological activity of the free (non-salt) compound and which can be administered as drugs or pharmaceuticals to an individual. A pharmaceutically acceptable salt intends ionic interactions and not a covalent bond. As such, an N-oxide is not considered a salt. Such salts, example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth metal ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al, Pharmaceutical Salts, J. Pharm. Sci. 66(1):1-19, 1977. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrase or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc. suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

As used herein, and unless otherwise specified, the term “subject” is defined herein to include animals such as mammals, including, but not limited to, primates (e.g., humans), cows, sheep, goats, horses, dogs, cats, rabbits, rats, mice and the like. In specific embodiments, the subject is a human.

### B. HETEROCYCLIC COMPOUNDS AND PHARMACEUTICAL COMPOSITIONS THEREOF

### B.1. Representative Compounds

Some representative compounds of the invention are listed in Table 1.

TABLE 1
Representative compounds of the invention

| Cpd | |
|---|---|
| ID # | Structures |
| a | 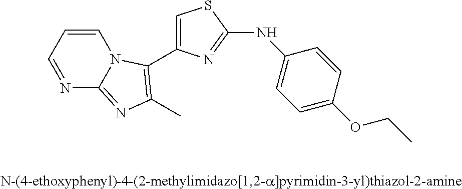 |
| b | 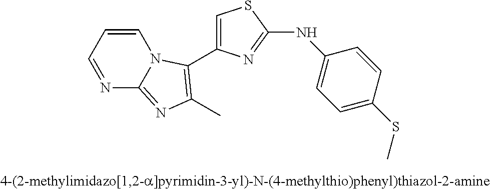 |
| c | 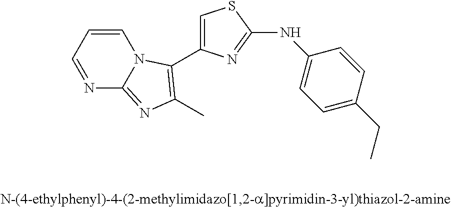 |
| d | 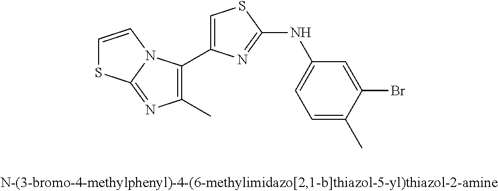 |
| e | 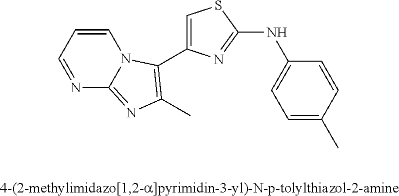 |
| f | 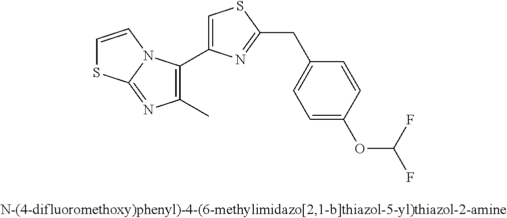 |
| g | 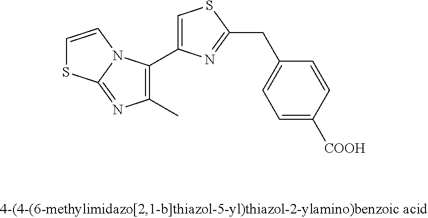 |
| h | 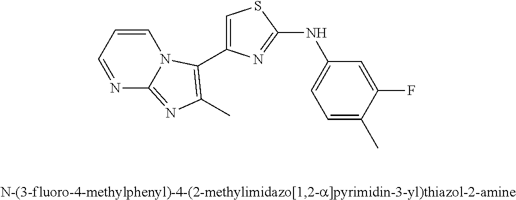 |
| i | 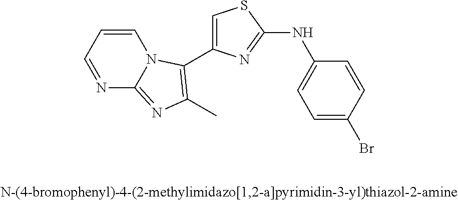 |
| j | 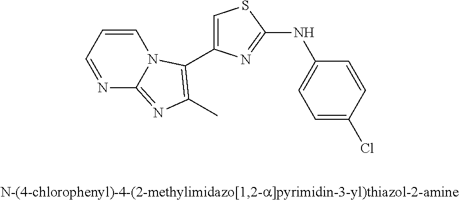 |
| k | 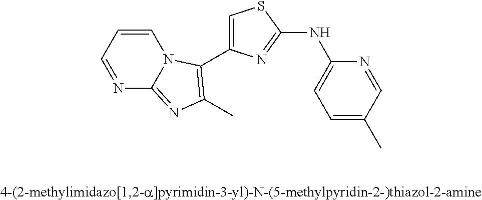 |
| l | 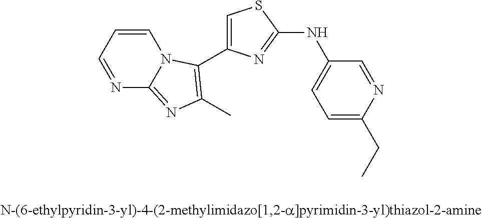 |
| m | 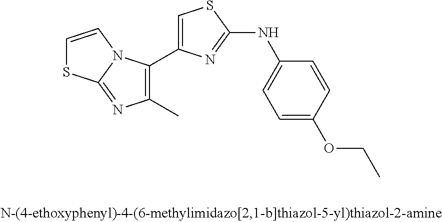 |
| n | 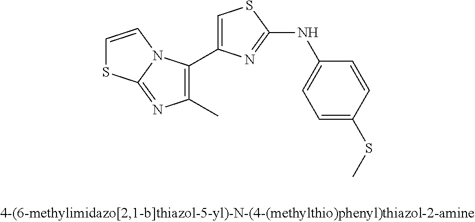 |
| o | 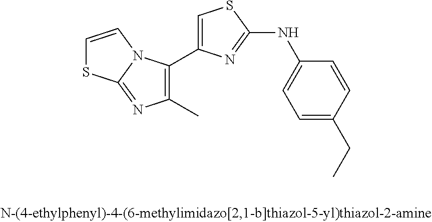 |
| p | 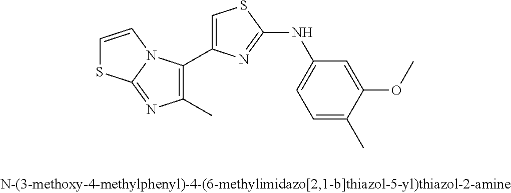 |
| q | 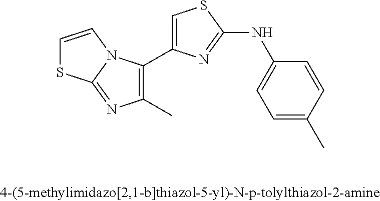 |
| r | 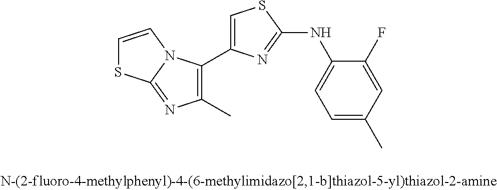 |
| s | 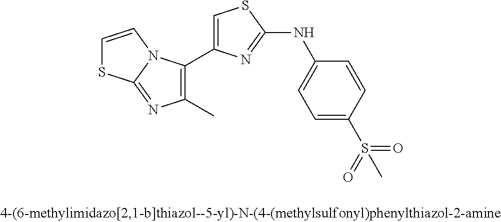 |
| t | 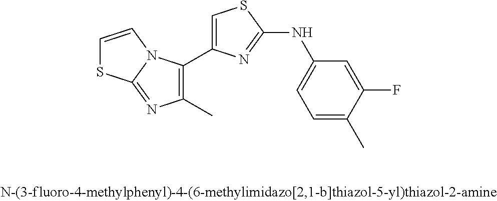 |
| u | 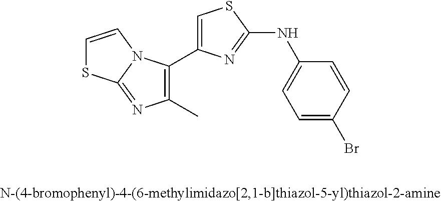 |
| v | 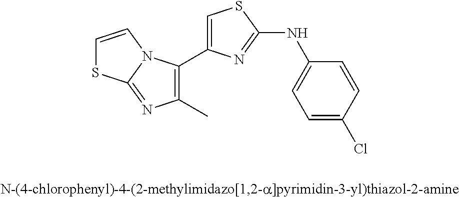 |
| w | 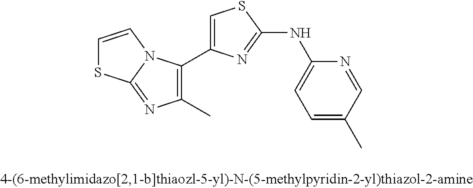 |
| x | 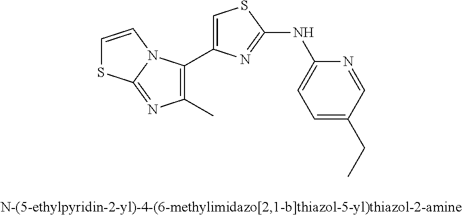 |

### B.2. Exemplary Synthetic Methods

The exemplary compounds were synthesized by routes as illustrated in Schemes I and II. Known methods from the art can be used and modified by those skilled in the art to produce the compounds of the invention from available starting materials. Some useful methods are disclosed in published PCT application WO 2009/023402. Additional synthesis methods that may be useful for preparing compounds within the scope of the invention are disclosed, for example, in Hayakawa et al., Biorg. Med. Chem. Vol. 15, 403-12 (2007); Ermolat'ev, et al., J. Comb. Chem. Vol. 8, 659-63 (2006); Carballares, et al., Tetrahedron Lett. vol. 48, 2041-45 (2007); and Rupert, et al., Biorg. Med. Chem. Lett., vol. 13, 347-50 (2003).

Schemes below provide exemplary synthetic methods for the preparation of the compounds provided herein. One of ordinary skills in the art will understand that similar methods may be employed to prepare the compounds provided herein. In other words, one of ordinary skills in the art will recognize that suitable adjustments to reagents, protecting groups, reaction conditions, and reaction sequences may be employed to prepare a desired embodiment. The reactions may be scaled upwards or downwards to suit the amount of material to be prepared.

Specific schemes for preparing compounds provided herein are shown below. Detailed reaction conditions are provided for various specific examples herein below. One of ordinary skills of the art will understand that the following schemes may be modified with appropriate reagents, protecting groups, conditions, starting materials, or reaction sequences to suit the preparation of other embodiments provided herein.

All references disclosed herein are incorporated by reference in their entireties.

Compound names were generated with ChemDraw Ultra 10.0; and intermediates and reagent names used in the examples were also generated with ChemDraw Ultra 10.0

2a. General Synthetic Scheme/Method for Compounds of Formula I:

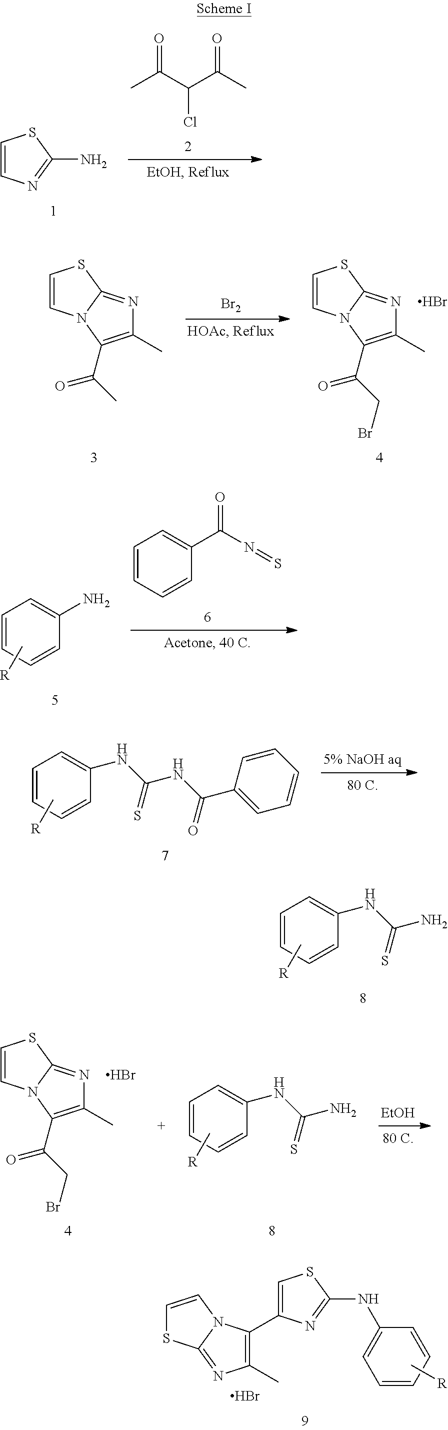

Thiazol-2-amine 1 was treated with 3-chloro pentane-2,4-dione 2 in EtOH at refluxing temperature to give the cyclized product 3, which was further brominated with bromine in HOAc to yield α-bromoketone 4. The substituted aniline 5 reacted with benzoyl isothiocyanate 6 in acetone to produce N-(phenyl carbamothioyl)benzamide 7, which was hydrolyzed with 5% aqueous NaOH at 80° C. to give substituted phenylthiourea 8. The two key intermediates 7 and 8 were then heated up in EtOH to form thiazole 9 (Formula I) as HBr salt in high yield, which could be converted to the other pharmaceutically acceptable salts (e.g. HCl salt) or free base form for in vitro and in vivo studies.

The substituted anilines of formula 5 that are needed for preparation of the compounds of the invention are commercially available or can be synthesized by known methods.

2b. General Synthetic Scheme/Method for Compounds as Formula II:

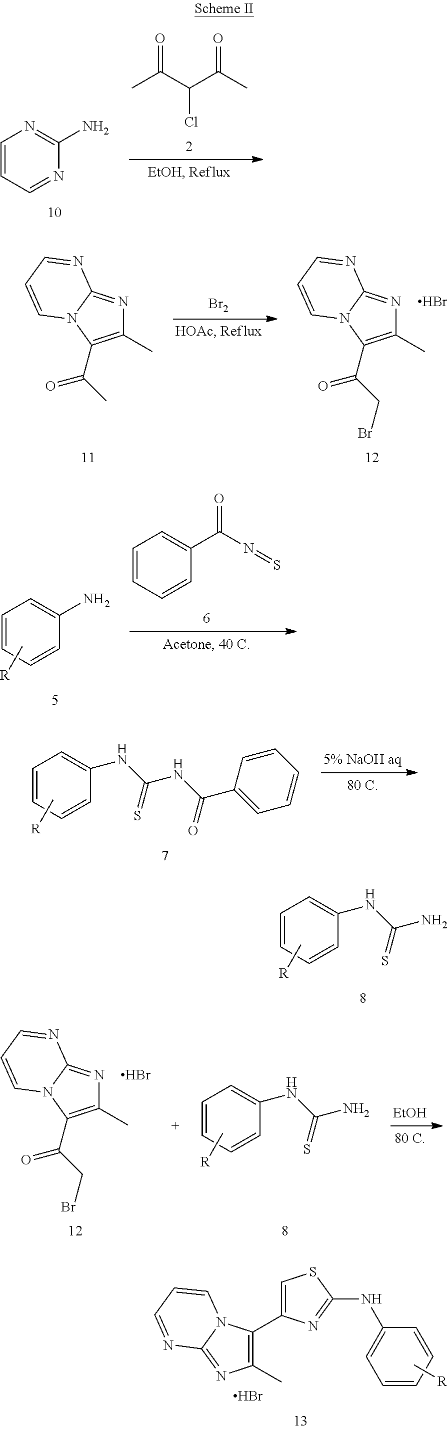

Pyrimidin-2-amine 10 was treated with 3-chloro pentane-2,4-dione 2 in EtOH at refluxing temperature to give the cyclized product 11, which was further brominated with bromine in HOAc to yield α-bromoketone 12. Substituted aniline 5 reacted with benzoyl isothiocyanate 6 in acetone to produce N-(phenyl carbamothioyl)benzamide 7, which was hydrolyzed with 5% aqueous NaOH at 80° C. to give substituted phenylthiourea 8. The two key intermediates 12 and 8 were then heated up in EtOH to form thiazole 13 (Formula II) as HBr salt in high yield, which could be converted to the other pharmaceutically acceptable slats (e.g. HCl salt) or free base form for in vitro and in vivo studies.

The substituted anilines of formula 5 that are needed for preparation of the compounds of the invention are commercially available or can be synthesized by known methods.

### B.3. Examples

The following Examples are provided to illustrate but not to limit the invention.

Following the synthetic methods/schemes described in Schemes I and II, the compounds in Table 1 were synthesized. Some exemplary syntheses are provided herein.

### Example B(1)

### Synthesis of 2-(4-ethoxyphenylamino)-4-(2-methyl-imidazo[1,2-a]pyrimidin-3-yl)thiazole monohydrobromide (a)

The synthesis for compound a is shown in Scheme III:

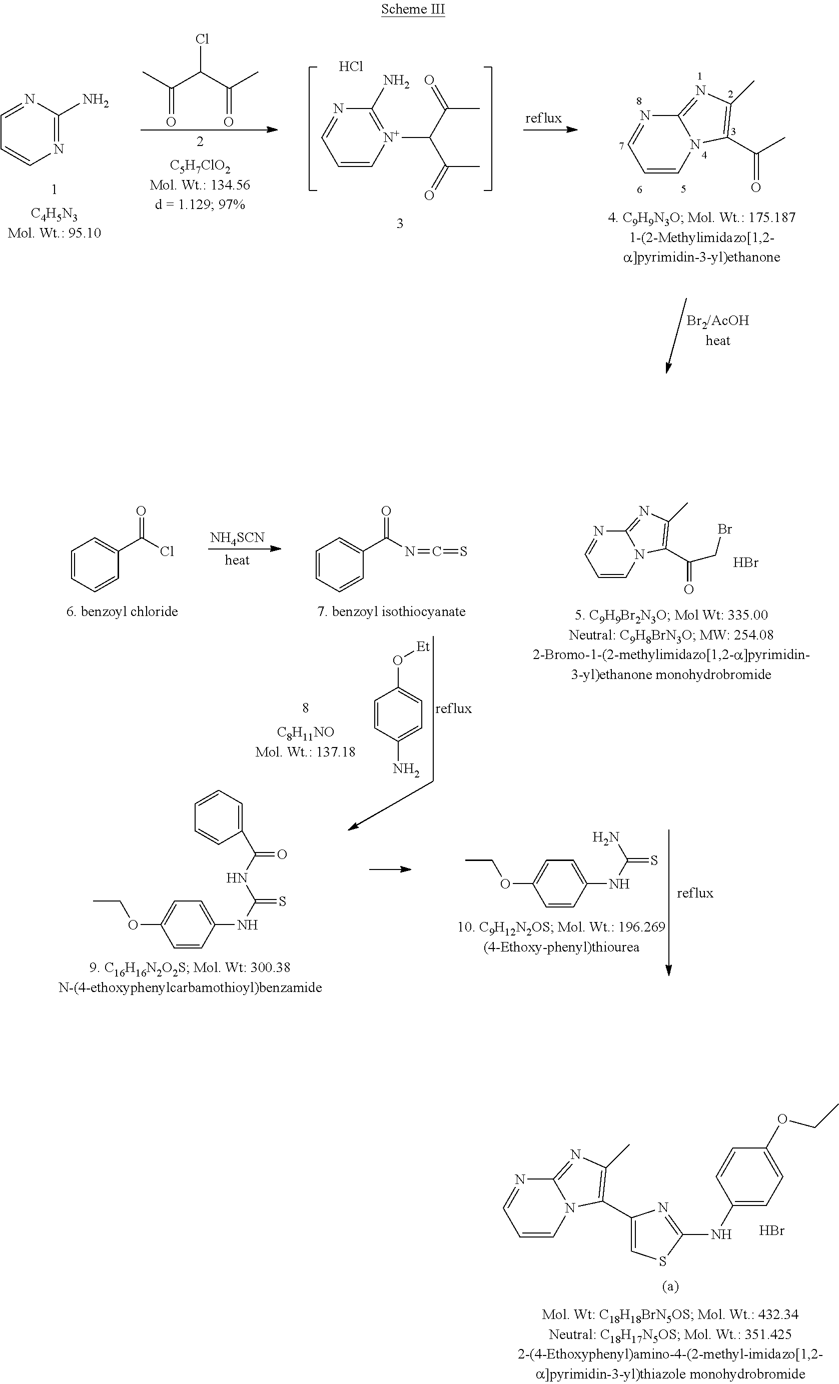

### Synthesis of 1-(2-methylimidazol[1,2-a]pyrimidin-3-yl) ethanone (4)

3-Chloro-2,5-pentanedione (2) (106 mL, 119 g, 887 mmol, 1.2 eq) was dissolved in 650 mL of anhydrous ethanol. 2-Aminopyrimidine (1) (71.5 g×97%=69.36 g, 729 mmol) was added to above stirred solution. The resulting mixture was refluxed for 40 h at an oil bath temperature of 100-105° C. The black reaction mixture was cooled and concentrated under reduced pressure. The residue was treated with saturated sodium bicarbonate solution (˜500 mL) in portions, and swelled the flask to mix well. The mixture was extracted with dichloromethane (×6). The extracts were washed with sodium bicarbonate solution and brine. The organic phase was dried and concentrated. The residue was purified by flash chromatography on a silica gel column (7×30 cm) by gradient elution using n-hexane-ethyl acetate (3:1, 2:1, 1:1, 1:2 and 0:1) and then dichloromethane-methanol (30:1, 20:1, 10:1 and 5:1). The product fractions were collected (TLC, R_{f} 0.36, 100% ethyl acetate) and concentrated providing a light black solid. Other fractions contained products were collected and re-purified again by the same way. 27.84 g (21.8%) of the final product was obtained. Portion of the product was re-crystallized from small amount of acetonitrile to give red to light brown crystals 4, m.p. 255.6-256.6° C. ¹H NMR (CDCl₃) δ 2.65 (s, 3H, 3-COCH₃), 2.86 (s, 3H, 2-CH₃), 7.04-7.12 (m, 1H, 6-H), 7.70-7.74 (m, 1H), 9.96-10.00 (m, 1H).

### Synthesis of 2-bromo-1-(2-methylimidazo[1,2-a]pyrimidin-3-yl)ethanone monohydrobromide (5) (bromination)

1-(2-methylimidazol[1,2-a]pyrimidin-3-yl)ethanone (4) (1.75 g, 10 mmol) was dissolved in 20 mL of glacial acetic acid by gently warming the flask, which was then cooled to room temperature. A solution of bromine (0.6 mL, 1.85 g, 11.5 mmol, 1.15 eq) in 4 mL of acetic acid was added slowly to the above stirred reaction mixture at room temperature for more than 30 min [Caution: bromine is highly corrosive. Handling bromine needs to be done very carefully in the well ventilated fume hood. Long gloves or double gloves are needed for operator. Absolutely avoid splash onto skin or breathe the vapor]. Some solid precipitated out before completing the addition. The reaction mixture was stirred at an oil bath temperature of 100-110° C. for 3 hours, and then stirred at room temperature overnight. The solid was filtered and washed several times with acetone, occasionally washed with anhydrous ethanol in the middle of acetone wash. The light brown solid was taken up with acetone containing small amount of ethanol, and the mixture was stirred at room temperature for more than 5 hours. The solid was filtered, and the solid was washed as mentioned above (one more taken up and wash cycle is recommended for larger scale). After dried under vacuum, 2.43 g (72.5%) pale brown powder solid product 5 was obtained as the monohydrobromide salt. Decomposed at above 250° C. TLC, R_{f} 0.42 (100% ethyl acetate). ¹HNMR (DMSO-d₆) δ 2.82 (s, 3H, 2-CH₃), 4.83 (s, 2H, CH₂Br), 7.40-7.50 (m, 1H), 8.80-8.90 (m, 1H), 9.82-9.90 (m, 1H).

### Synthesis of 1-benzoyl-3-[4-(ethoxyphenyl)]thiourea (9)

[References: ARKIVOC 2003, 434-442; Bioorg. Med. Chem. 2000, 2663]. Benzoyl chloride (6) (14.0 mL, 16.95 g, 120 mmol) was added dropwise at room temperature to a stirred solution of ammonium thiocyanate (10.26 g, 135 mmol, 1.125 eq) in 100 mL of acetone. Some white solid precipitated out. The reaction mixture was heated to reflux for 5 min (oil temperature ˜65-70° C.). Thus obtained benzoyl isothiocyanate (7) was used directly to the next step without purification. A solution of 4-ethoxyaniline (8) (17.0 mL, 18.1 g, 132 mmol, 1.1 eq) in 25 mL of acetone was added slowly to the above stirred reaction mixture while it is still in the oil bath (65-70° C.). The addition needs to be done very slowly for ˜1 h considering the exothermic reaction. A lot of white solid precipitated out. The reaction mixture was swelled by hands and further refluxed for 5 min. The cooled reaction mixture was poured into ice water. The solid was filtered and washed 3 times with water. The solid was re-crystallized from ethanol (˜1.6 L) to provide the desired product 9 as light yellow, long needles, yield 36 g (99%), m.p. 151.0-153.5° C.

### Synthesis of (p-ethoxyphenyl)thiourea (10)

Sodium hydroxide aqueous solution (1 M, 60 mL, 60 mmol, 1.2 eq) was added to a stirred mixture of 1-benzoyl-3-[4-(ethoxyphenyl)]thiourea (9) (16.5 g, 55 mmol) in 350 mL of ethanol. The reaction mixture was refluxed for 1 h, cooled and concentrated. The white solid was treated with water (˜200 mL). The solid was filtered and washed with water. The crude crystalline product was re-crystallized from ethanol, filtered and dried under vacuum providing 7.66 g (71.0%) desired product 10, m.p. 176.5-178.5° C. TLC, R_{f} 0.45 (n-hexane-ethyl acetate: 1:1). ¹HNMR (DMSO-d₆) δ 1.31 (t, 3H, J=6.8 Hz), 4.00 (q, 2H, J=6.8 Hz), 6.80-6.90 (m, 2H), 7.15-7.25 (m, 2H), 9.50 (s, 1H, NH).

### Synthesis of 2-(4-ethoxyphenylamino)-4-(2-methyl-imidazo[1,2-a]pyrimidin-3-yl)thiazole monohydrobromide (a) (cyclization into thiazole ring)

A mixture of 2-bromo-1-(2-methylimidazo[1,2-a]pyrimidin-3-yl)ethanone monohydrobromide (5) (2.43 g, 7.25 mmol) and (p-ethoxyphenyl)thiourea (10) (1.40 g, 7.1 mmol) in 140 mL of anhydrous ethanol was refluxed under stirring for 15 h (oil bath temperature ˜105° C.). It was then stirred at room temperature for 6 h or overnight. The solid was filtered and washed with acetone. The crude soft crystals were taken up with acetone-ethanol (3:1) and stirred at room temperature for more than 6 h or overnight. The solid was filtered and washed as above. The crude product was taken up with acetone-ethanol (3:1) and stirred at room temperature for more than 6 h. The crude product was filtered, washed, and re-crystallized from methanol. The methanol solution was filtered while hot to remove black dust, and then heated into solution. The yellow crystals was filtered and washed. It was re-crystallized two more times from methanol, and dried under vacuum to provide the desired product 11 as long, soft yellow needles, yield 1.55 g (50.5%), decomposed at above 240° C. TLC R_{f} 0.32 (dichloromethane-methanol: 20:1); R_{f} 0.46 (dichloromethane-methanol containing: 20:1 containing 1% ammonium hydroxide aqueous solution); R_{f} 0.30 (100% ethyl acetate X2). HPLC purity: 99%. ¹HNMR (DMSO-d₆) δ 1.32 (t, 3H, J=6.8 Hz), 2.67 (s, 3H), 4.00 (q, 2H, J=6.8 Hz), 6.90-6.95 (m, 2H), 7.35 (s, 1H), 7.49-7.52 (m, 2H), 7.61-7.67 (m, 1H), 8.94-8.97 (m, 1H), 9.57 (d, 1H, J=6.8 Hz), 10.28 (s, 1H, NH). ESI-MS, m/z 352 (M+1)⁺.

### Example B(2)

### Synthesis of 2-(4-bromophenyl)amino-4-(6-methylimidazo[2,1-b]thiazol-5-yl)-thiazole monohydrobromide (u)

The synthesis of compound u is shown in Scheme IV:

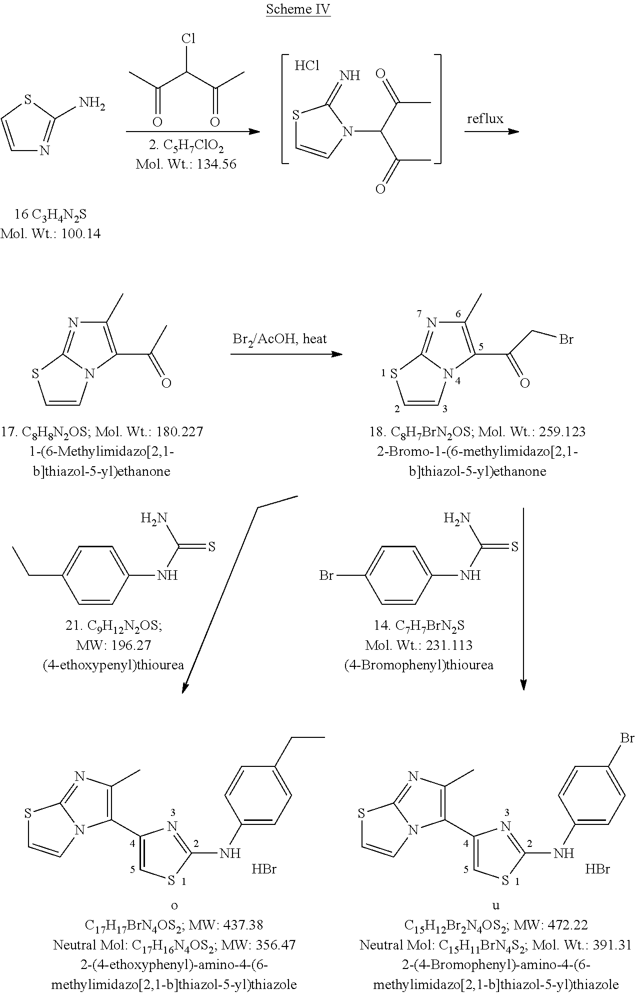

Synthesis of (p-bromophenyl)thiourea (14): similar method as the synthesis of compound 10 using p-bromo aniline instead of p-ethoxyl aniline.

Synthesis of 1-(6-Methylimidazo[2,1-b]thiazol-5-yl)ethanone (17). 2-Aminothiazole was recrystallized from anhydrous ethanol, filtered and dried before use. A solution of 2-aminothiazole (16) (20.9 g, 202.4 mmol.) and 3-Chloro-2,5-pentanedione (2) (33.7 g, 97%, 242.9 mmol, 1.2 eq) in 180 mL of anhydrous ethanol was refluxed for 72 h in an oil bath. The black reaction mixture was cooled and concentrated under reduced pressure. The residue was treated with saturated sodium bicarbonate solution in portions, and then extracted with dichloromethane. The organic phase was dried and concentrated. The residue was purified by flash chromatography on a silica gel column using dichloromethane-methanol (80:1). The product fractions were collected (TLC, R_{f} 0.60 neutral form; R_{f}=0.5 salt form, dichloromethane-methanol 40:1) and concentrated providing white solid product 17 in 11.7% yield, 1.6 g neutral form and 3.2 g salt form. ¹H NMR (CDCl₃) δ 2.55 (s, 3H, 5-COCH₃), 2.70 (s, 3H, 6-CH₃), 6.78 (d, 1H, J=4.8 Hz), 8.39 (d, 1H, J=4.8 Hz).

Synthesis of 2-bromo-1-(6-methylimidazo[2,1-b]thiazol-5-yl)ethannone hydrobromide (18). 1-(6-Methylimidazo[2,1-b]thiazol-5-yl)ethanone (17) (0.54 g, 3.0 mmol) was dissolved in 7 mL of glacial acetic acid. A solution of bromine (0.18 mL, 0.56 g, 3.5 mmol) in 3 mL of glacial acetic acid was added slowly to above stirred solution in 30 min. Some yellow solid appeared. The mixture was heated to reflux under stirring for 3 h, and then stirred at room temperature overnight. The solid was filtered and washed three times with acetone, and stirring for 3-5 h for each wash was needed. The solid was filtered and dried under vacuum to provide 0.73 g (71.6%) white solid the desired product 18.

Synthesis of 2-(4-bromophenyl)amino-4-(6-methylimidazo[2,1-b]thiazol-5-yl)-thiazole monohydrobromide (u). A mixture of 2-bromo-1-(6-methylimidazo[2,1-b]thiazol-5-yl)ethannone hydrobromide (18) (0.73 g, 2.0 mmol) and (p-bromophenyl)thiourea (14) (0.50 g, 2.0 mmol) in 10 mL of anhydrous ethanol was refluxed under stirring for 20 h, and then cooled to room temperature. The solid was filtered. The crude white solid product was re-crystallized from methanol. The methanol solution was filtered while still hot to remove possible dust, and then heated into solution. It was re-crystallized two more times from methanol, and dried under vacuum to provide the desired product u as a white solid, yield 0.34 g (36%), HPLC purity 98.49%, mp>250° C. ¹HNMR (CD₃OD) δ 2.68 (s, 3H), 7.45 (s, 1H), 7.18 (s, 1H), 7.18-7.47 (m, 2H), 7.54-7.66 (m, 2H), 7.66 (d, 1H, J=4.4 Hz), 8.94 (d, 1H, J=4.4 Hz).

### Example B(3)

### Synthesis of 2-(4-ethylphenyl)amino-4-(6-methylimidazo[2,1-b]thiazol-5-yl)-thiazole monohydrobromide (o)

The synthesis of compound (o) is shown in Scheme IV.

Synthesis of (p-bromophenyl)thiourea (21): similar method as the synthesis of compound 10 using p-ethyl aniline instead of p-ethoxyl aniline.

Synthesis of 2-(4-ethylphenyl)amino-4-(6-methylimidazo[2,1-b]thiazol-5-yl)-thiazole monohydrobromide (o). A mixture of 2-bromo-1-(6-methylimidazo[2,1-b]thiazol-5-yl)ethanone hydrobromide (18) (20 g, 91%, 53.5 mmol) and (p-ethylphenyl)thiourea (10) (10.1 g, 56.2 mmol) in 40 mL of anhydrous ethanol was refluxed under stirring for 3 h, and then cooled to room temperature. The solid was filtered and washed with ethanol. The crude white solid product was re-crystallized from methanol. The methanol solution was filtered while still hot to remove possible dust, and then heated into solution. It was re-crystallized one more time from methanol, and dried under vacuum to provide the desired product (o) as a white solid. This HBr salt was converted to free base, and then to HCl salt which was re-crystallized from 50% ethanol/water to yield off-white crystal product (14.8 g, 73.4%). HPLC purity 99%, mp=181-183° C. ¹HNMR (CD₃OD) δ 1.11 (t, 3H, J=7.6 Hz), 2.52 (s, 3H), 2.58 (m, 2H), 7.11 (d, 2H, J=8 Hz), 7.22 (s, 1H), 7.52 (d, 2H, J=8.4 Hz), 7.75 (d, 1H, J=4.4 Hz), 8.42 (d, 1H, J=4.4 Hz). ESI-MS m/z 341.5 (M+1)⁺.

Preparation of Free Base Compounds from Compounds of Formula I or II (HBr Salt).

The HBr salt was suspended in methanol, and the excess amount of sodium bicarbonate was added with vigorous stirring until the suspended compound salt was completely dissolved. Excess amount of inorganic salt was filtered off. The solution was concentrated, and the residue was re-crystallized from methanol or ethanol or ethanol/water or any other organic solvent(s) or mixture of solvents to provide crystalline material as the free base compound.

Preparation of different salts. The free base compound obtained and 1.1 equivalent amount of the selected acids. The solution was concentrated, and the residue was recrystallized from methanol or ethanol or ethanol/water or any other organic solvent(s) or mixture of solvents to provide crystalline material/polymorphic substances of the desired salts with the selected anions as described above.

All possible crystal forms/polymorphs of the free bases or salts of the compounds described are covered with this invention.

### B.3. Additional Active Agents

The compounds herein can be combined with other pharmacologically active compounds (“additional active agents”) in methods and compositions of the invention. It is believed that certain combinations work synergistically in the treatment of particular types of cancer and certain diseases and conditions associated with, or characterized by, undesired angiogenesis. Immunomodulatory compounds can also work to alleviate adverse effects associated with certain second active agents, and some second active agents can be used to alleviate adverse effects associated with immunomodulatory compounds.

One or more active ingredients or agents can be used in the methods and compositions of the invention together. Additional active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions of the invention include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

This invention encompasses the use of native, naturally occurring, and recombinant proteins. The invention further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with compounds of the invention include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. Compounds of the invention can also be combined with, or used in combination with, anti-TNF-.alpha.antibodies.

Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits of the invention. See, e.g., Emens, L. A., et al., Curr. Opinion Mol. Ther. 3(1):77-84 (2001).

In one embodiment of the invention, the large molecule active agent reduces, eliminates, or prevents an adverse effect associated with the administration of an immunomodulatory compound. Depending on the particular immunomodulatory compound and the disease or disorder begin treated, adverse effects can include, but are not limited to, drowsiness and somnolence, dizziness and orthostatic hypotension, neutropenia, infections that result from neutropenia, increased HIV-viral load, bradycardia, Stevens-Johnson Syndrome and toxic epidermal necrolysis, and seizures (e.g., grand mal convulsions). A specific adverse effect is neutropenia.

Additional active agents that are small molecules can also be used to alleviate adverse effects associated with the administration of an immunomodulatory compound. Examples of small molecule active agents include, but are not limited to, anti-cancer agents, antibiotics, immunosuppressive agents, and steroids.

Examples of anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific additional active agents include, but are not limited to, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

### B.4. Formulation

Any suitable formulation of the compounds described herein can be prepared. In cases where compounds are sufficiently basic or acidic to form stable nontoxic acid or base salts, administration of the compounds as salts may be appropriate. Examples of pharmaceutically acceptable salts are organic acid addition salts formed with acids that provide a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by contacting a sufficiently basic compound such as an amine with a suitable acid, affording a physiologically acceptable salt. Alkali metal (e.g., sodium, potassium or lithium) or alkaline earth metal (e.g., calcium) salts of carboxylic acids also are included, and are prepared by conventional methods.

The invention also includes pharmaceutical compositions comprising at least one compound of the invention admixed with at least one pharmaceutically acceptable excipient. Preferably, at least one such excipient is an excipient other than water or a C1-C3 alcohol or a dimethyl sulfoxide.

Compounds of the invention can be administered by conventional routes, including orally, topically, transdermally, or by inhalation or injection. The compounds of the invention can be formulated by those skilled in the art by reference to known methods, and the formulation can be tailored according to the intended route of administration. Suitable methods for formulating organic compounds are described, for example, in REMINGTON'S PHARMACEUTICAL SCIENCES, 18^{th} ed. (1990), which is incorporated herein by reference.

Where these compounds are administered in a pharmacological composition, it is contemplated that the compound can be formulated in admixture with a pharmaceutically acceptable carrier. For example, contemplated compounds can be administered orally as pharmacologically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds having formula I or II as described herein are generally soluble in organic solvents such as chloroform, dichloromethane, ethyl acetate, ethanol, methanol, isopropanol, acetonitrile, glycerol, N,N-dimethylformamide, N,N-dimethylacetamide, dimethylsulfoxide, etc. In one embodiment, the present invention provides formulations prepared by mixing a compound having formula I-II with a pharmaceutically acceptable carrier. In one aspect, the formulation may be prepared using a method comprising: a) dissolving a described compound in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of suitable alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### B.5. Pharmaceutical Compositions

The present invention also has the objective of providing suitable topical, oral, systemic and parenteral pharmaceutical formulations for use in the novel methods of treatment of the present invention. The compositions containing compounds of this invention as the active ingredient can be administered in a wide variety of therapeutic dosage forms in vehicles for oral, systemic or targeted site administration.

The present invention also provides pharmaceutical compositions comprising one or more compounds of this invention in association with a pharmaceutically acceptable carrier. Preferably these compositions are in unit dosage forms such as tablets, pills, capsules, powders, granules, suspensions, gels, softgels, sterile parenteral solutions, emulsions, aerosol, liquid sprays, drops, ampoules, autoinjector devices or suppositories; for oral, parenteral, intranasal, sublingual or rectal administration, or for administration by inhalation or insulation.

For preparing solid compositions such as tablets or capsules, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid composition containing a homogenous mixture of a compound of the present invention, or a pharmaceutically acceptable salt thereof. Furthermore, the principal active ingredient can mix with one or more pharmaceutical carriers to provide a dosage form with improved bioavailability or other pharmacokinetic properties. Example of such systems include but not limited to: Spray Dried Dispersion solid dosage form with ingredients such as hydroxypropyl methylcellulose (HPMC), hypromellose acetate succinate (HPMCAS); Nano-particles formulation with ingredients such as Low viscosity hydroxypropylcellulos (HPC-SL), docusate sodium, pluronics, phosphatidylcholine, lecithin and cholesterol; Lipid-base formulation with ingredients such as phosphatidylcholine, polyvinylpyrrolidone, lecithin, and cholesterol; Cyclodextrine formulation with ingredients such as sulfobutylether-beta-cyclodextrin (SBECD) and 2-hydroxypropyl-beta-cyclodextrin (HPCD). When referring to these compositions as homogeneous, it is meant that the active ingredient is dispersed evenly throughout the composition so that the composition may be readily subdivided into equally effective unit dosage forms such as tablets, pills and capsules and lipid-based formulation

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. An enteric layer can separate the two components. That enteric layer serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol and cellulose acetate.

The liquid forms in which the novel compositions of the present invention may be incorporated for administration orally or by injection include aqueous solutions, suitably flavoured syrups, aqueous or oil suspensions, emulsions with edible oils such as cottonseed oil, sesame oil, coconut oil or peanut oil, microemulsions or self-emulsifying systems with surfactant or co-solvent such as polysorbate 80, tocopheryl polyethylene glycol succinates (TPGS), Cremophor, capmul MCM, polyethylene glycol; liposome or nanoparticle formulation with ingredients such as phosphatidylcholine, cholesterol, lecithin, HPC-SL, Docusate Soldium; Cyclodextrine complex formulation with ingredients such as SBECD, HPCD to enhance solubility. Suitable dispersing or suspending agents for aqueous suspensions include synthetic and natural gums such as dextran, sodium carboxymethylcellulose, methylcellulose, polyvinyl-pyrrolidone or gelatin.

Compounds of this invention may be administered in any of the foregoing compositions and according to dosage regimens that is effective in efficacy studies.

Compounds of the present invention may be used alone at appropriate dosages defined by routine testing in order to obtain optimal anticancer effect. In addition, co-administration or sequential administration of other oncology agents is desirable.

### C. METHODS OF USING THE NOVEL COMPOUNDS AND PHARMACEUTICAL COMPOSITIONS

### C.1. Methods of Using the Novel Compounds

The compounds of the present invention can be used as cytotoxic and/or cytostatic agents in treating cancers or other types of proliferative disease. These compounds may function through any type of action mechanisms. For example, the compounds may inhibit molecules and/or signal transduction pathways leading to arrest of the cell cycle at G2/M phase, which might eventually induce apoptosis in tumor cells (see, e.g., Weung et al. (1997) Biochim. Biophys. Res. Comm., vol: 263, pp 398-404). In another example, the compounds may disturb tubulin assembly/disassembly, which may inhibit the cell mitosis and induce the cell apoptosis (see, e.g., Panda et al., (1997) Proc. Natl. Acad. Sci. USA, vol: 94, 10560-10564). The compounds may also inhibit endothelial cell proliferation and angiogenesis effect (see, e.g., Witte et al., 1998, Cancer Metastasis Rev. vol. 17: 155-161). The compounds of the invention are shown to be active on various cancer cell lines.

In another aspect, the present invention is directed to a method of treatment of cancers of all tissue or organ origin including but not limited to sarcoma, epidermoid cancer, fibrosarcoma, cervical cancer, leukemia, lymphoma, lung cancer, non-small cell lung cancer, colon cancer, CNS cancer, melanoma, ovarian cancer, renal cancer, prostate cancer, breast cancer, head and neck cancers, pancreatic cancer and other types of proliferative disease in a mammal comprising administering a therapeutically effective amount of compound having Formula I-II as a cytotoxic and/or cytostatic agent to said subject in need of such treatment, in at least one treatment.

In yet another aspect, the present invention is directed to a method for manufacturing a pharmaceutical preparation for the treatment of cancers of all tissue or organ origin including but not limited to leukemia, lymphoma, lung cancer, colon cancer, CNS cancer, melanoma, ovarian cancer, renal cancer, prostate cancer or breast cancer, and other types of a proliferative disease, comprising admixing a therapeutically effective amount of a compound having Formula I-II with a pharmaceutically acceptable carrier.

To practice the method of the present invention, compounds having Formulas I-II and pharmaceutical compositions thereof may be administered orally, parenterally, by inhalation spray, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. In some embodiments, the compounds of the invention are delivered by injection, i.e., parenterally. In some embodiments, the preferred route of administration is by intravenous or intraperitoneal injection.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

An effective amount of a compound of the invention can be determined by routine experimentation as is known in the art. Typically, this involves administration of an amount shown to be well tolerated, and gradually increasing the dosage until a desired effect is achieved, such as reduction in symptoms, reduction n tumor size, or cessation of tumor growth. In some embodiments a starting dosage of about 5-10 mg/kg is used, and the dosage is increased incrementally once per week by about 50% each time until a desired effect is noted or tolerance problems are observed. In some embodiments, a suitable dosage is between about 5 and 250 mg/kg; or between about 10 and 150 mg/kg. Dosages between 10 and 100 mg/kg are sometimes preferred. Dosing can be done once, once weekly, once daily or more than once daily. In some embodiments, 1-4 doses are delivered per day to a subject in need of treatment.

In addition, the compounds having formula I-II may be administered alone or in combination with other anticancer agents for the treatment of various cancers or conditions. Combination therapies according to the present invention comprise the administration of at least one compound of the present invention or a functional derivative thereof and at least one other pharmaceutically active ingredient. The active ingredient(s) and pharmaceutically active agents may be administered separately or together. The amounts of the active ingredient(s) and pharmaceutically active agent(s) and the relative timings of administration will be selected in order to achieve the desired combined therapeutic effect.

In yet another aspect, the present invention is directed to a method of treatment of restenosis after coronary stenting for patients with coronary artery diseases with a compound having formula I-II.

The main cause of restenosis after coronary stenting for patients with coronary artery disease is neointimal hyperplasia resulting from the proliferation and migration of smooth-muscle cells and extracellular matrix productions (see, for example, “Pathology of acute and chronic coronary stenting in humans”, by Farb, A., Sangiorgi, G., Certer, A. J., et al, in Circulation, vol. 99, pp 44-52, 1999). Compounds that have anti-proliferation capability may have an effect in reducing the risk of clinical and angiographic restenosis when such compounds are delivered with a suitable means (see, for example, “A polymer-based, paclitaxel-eluting stent in patients with coronary artery disease”, by Stone, G. W., Ellis, S. G., Cox, D. A, et al, in New England Journal of Medicine, vol. 350: pp 221-231, 2004). Thus, with compounds having formula I-IX in treating tumor, they may be also useful in inhibiting proliferation of the cells involved in neointimal hyperplasia and thus reducing the incidence of neointimal hyperplasia and restenosis. Various methods may be used in delivering effectively the compounds to these cells. For example, a composition comprising above-described compounds having formula I-X can be administered orally, parenterally, or via an implanted reservoir. In other examples, the approaches described in the following papers may also be used: “A polymer-based, paclitaxel-eluting stent in patients with coronary artery disease”, by Stone, G. W., Ellis, S. G., Cox, D. A. et al, in New England Journal of Medicine, vol. 350: pp 221-231, 2004; “A randomized comparison of a sirolimus-eluting stent with a standard stent for coronary revascularization”, by Morice, M.-C., Serruys, P. W., Sousa, J. E., et al, in New England Journal of Medicine, vol. 346: pp 1773-1780, 2002; “Sirolimus-eluting stents versus standard stents in patients with stenosis in a native coronary artery”, by Moses, J. W., Leon, M. B., Popma, J. J., et al, in New England Journal of Medicine, vol. 349: pp 1315-1323, 2003.

### Methods of Treatments and Prevention

Methods of this invention encompass methods of treating, preventing and/or managing various types of cancer. As used herein, unless otherwise specified, the term “treating” refers to the administration of a compound of the invention or other additional active agent. As used herein, unless otherwise specified, the term “preventing” refers to the administration prior to the onset of symptoms, particularly to patients at risk of cancer. The term “prevention” includes the inhibition of a symptom of the particular disease or disorder. Patients with familial history of cancer are preferred candidates for preventive regimens. As used herein and unless otherwise indicated, the term “managing” encompasses preventing the recurrence of the particular cancer in a patient who had suffered from it, and/or lengthening the time a patient who had suffered from the cancer remains in remission.

As used herein, the term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation; in particular, refractory to thalidomide.

### C.2. Methods of Treatments and Prevention

Methods of this invention encompass methods of treating, preventing and/or managing various types of cancer. As used herein, unless otherwise specified, the term “treating” refers to the administration of a compound of the invention or other additional active agent. As used herein, unless otherwise specified, the term “preventing” refers to the administration prior to the onset of symptoms, particularly to patients at risk of cancer. The term “prevention” includes the inhibition of a symptom of the particular disease or disorder. Patients with familial history of cancer are preferred candidates for preventive regimens. As used herein and unless otherwise indicated, the term “managing” encompasses preventing the recurrence of the particular cancer in a patient who had suffered from it, and/or lengthening the time a patient who had suffered from the cancer remains in remission.

As used herein, the term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation; in particular, refractory to thalidomide.

### C.3. Biological Screening and Anticancer Activity:

### In Vitro Cell-Based Screening Using Real-Time Cell Electronic Sensing (RT-CES)

The biological activity of compounds disclosed herein was monitored and profiled using the Real-Time Cell Electronic Sensing (RT-CES®) system from ACEA Biosciences, Inc. The RT-CES system utilizes cell-substrate impedance technology to monitor cellular behavior inside tissue culture wells in a microtiter plate format. The technology features in the integration of molecular and cell biology with microelectronics and is based on the electronic detection of biological assay process. The details of this cell electronic sensing technology and associated devices, systems and methods of use are described in U.S. Pat. No. 7,167,585; U.S. Pat. No. 7,468,255; PCT publication number WO 2004/010102; U.S. Pat. No. 7,470,533; and U.S. Pat. No. 7,459,303, each of which is incorporated herein by reference. Additional details of RT-CES technology are further disclosed in U.S. Pat. No. 7,468,255.

For measurement of cell-substrate or cell-electrode impedance using RT-CES technology, microelectrodes having appropriate geometries are fabricated onto the bottom surfaces of microtiter plate or similar device, facing into the wells. Cells are introduced into the wells of the devices, and make contact to and attach to the electrode surfaces. The presence, absence or change of properties of cells affects the electronic and ionic passage on the electrode sensor surfaces. Measuring the impedance between or among electrodes provides important information about biological status of cells present on the sensors. When there are changes to the biological status of the cells analogue electronic readout signals are measured automatically and in real time, and are converted to digital signals for processing and for analysis. In a RT-CES system, a cell index (arbitrary representation of change in impedance) is automatically derived and provided based on measured electrode impedance values. The cell index obtained for a given well reflects: 1) how many cells are attached to the electrode surfaces in this well; 2) how well cells are attached to the electrode surfaces in this well. Thus, the more the cells of same type in similar physiological conditions attach the electrode surfaces, the larger the cell index. And, the better the cells attach to the electrode surfaces (e.g., the cells spread-out more to have larger contact areas, or the cells attach tighter to electrode surfaces), the larger the cell index.

The RT-CES system comprises three components, an electronic sensor analyzer, a device station and 16× or 96× microtiter devices. Microelectrode sensor array was fabricated on glass slides with lithographical microfabrication methods and the electrode-containing slides are assembled to plastic trays to form electrode-containing wells. The device station receives the 16× or 96× microtiter plate devices and is capable of electronically switching any one of the wells to the sensor analyzer for impedance measurement. In operation, the devices with cells cultured in the wells are placed into a device station that is located inside an incubator. Electrical cables connect the device station to the sensor analyzer. Under the RT-CES software control, the sensor analyzer can automatically select wells to be measured and continuously conduct impedance measurements. The impedance data from the analyzer is transferred to a computer, analyzed and processed by the integrated software.

Impedance measured between electrodes in an individual well depends on electrode geometry, ionic concentration in the well and whether there are cells attached to the electrodes. In the absence of the cells, electrode impedance is mainly determined by the ion environment both at the electrode/solution interface and in the bulk solution. In the presence of the cells, cells attached to the electrode sensor surfaces will alter the local ionic environment at the electrode/solution interface, leading to an increase in the impedance. The more cells there are on the electrodes, the larger the increase in cell-electrode impedance. Furthermore, the impedance change also depends on cell morphology and the extent to which cells attach to the electrodes.

To quantify cell status based on the measured cell-electrode impedance, a parameter termed Cell Index is derived. Cell Index is a quantitative measure of the status of the cells in an electrode-containing well. Under the same physiological conditions, more cells attached on to the electrodes leads to larger cell-electrode resistance value, leading to a larger value for Cell Index. Furthermore, for the same number of cells present in the well, a change in the cell status such as morphology will lead to a change in the Cell Index. For example, an increase in cell adhesion or cell spreading leads to larger cell-electrode contact area which will lead to an increase in cell-electrode resistance and thus a larger value for Cell Index.

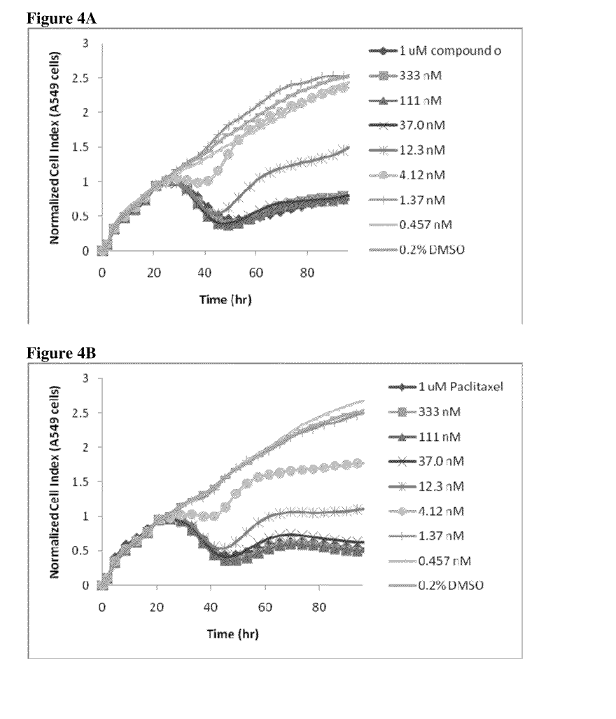

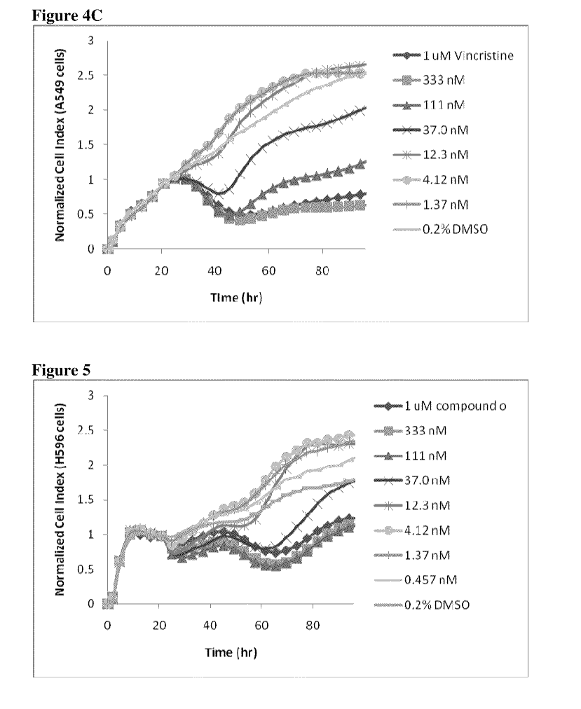

The interaction of biologically active compounds with cells growing inside the wells of the E-Plates results in unique activity patterns (i.e., unique cell impedance curves or cell index curves in response to a compound treatment) that is dependent on the biological mechanism of the compound itself, the concentration, length of incubation and the cell type. The “signature” cell responsive patterns to each compound correlates with specific biological phenomenon such as cell cycle arrest, morphology change and cell death. Cell response profiling on the RT-CES system has proven effective and we have shown that compounds with similar mechanism of action displays similar patterns. Thus, the similarity in the cell responsive patterns to compound treatment may indicate similarity in mechanism of action, mode of resistance and possibly molecular targets. We have identified a unique RT-CES signature pattern for cells undergoing mitotic arrest in response to treatment with anti-mitotic agents. As an example, FIGS. 4B and 4C show specific profile of A549 lung cancer cells treated with different concentrations of well know anti-mitotic agents paclitaxel and vincristine.

We evaluated the response of a number of cancer cell lines to some of the novel compounds using RT-CES system (ACEA Biosciences) or xCelligence system (Roche). Note that RT-CES system and xCelligence system are essentially same, employing the same ACEA real time cell impedance measurement technology. The time-dependent, cell responsive patterns of some of the invented compounds (at certain concentrations) were somewhat similar to those of paclitaxel and vincristine (at certain concentrations). Thus, these compounds may have mechanisms of anticancer action similar to those of paclitaxel and vinblastine. On the other hand, these compounds may act on cancer cells through other mechanisms of action, different from those of paclitaxel and vinblastine, even though the time-dependent, cell responsive patterns of these invented compounds are similar to those of paclitaxel and vincristine. It is also possible that these compounds act on cancer cells through multiple mechanisms of action, including the mechanism of action similar to those of paclitaxel and vinblastine.

We have tested the activity of the compounds of the invention as measured with the RT-CES methods described herein. The data show that the compounds are active on a wide range of cancer cell lines, and are much less active on normal cells. Here we use one of the active compounds, COMPOUND O, as an example. FIGS. 4-24 show the time-dependent cell index for a number of human cancer cell lines prior to and after addition of COMPOUND O at various concentrations. These cell lines include A549 (non-small cell lung cancer cell line), NCI-H460 (large cell lung carcinoma cell line), H1993 cells (non-small cell lung cancer cell line), H1838 cells (non-small cell lung cancer cell line), H2347 cells (non-small cell lung cancer cell line), SW620 cells (colon cancer cell line), GTL16 cells (gastric cancer cell line), HT29 cells (colon cancer cell line), A172 cells (brain cancer cell line), U138 cells (brain cancer cell line), U118 cells (brain cancer cell line), SW1088 cells (brain cancer cell line), HT1080 cells (connective tissue cancer cell line), B×PC3 cells (pancreatic cancer cell line), HepG2 cells (liver cancer cell line), SKOV3 cells (ovarian cancer cell line), MCF7 cells (breast cancer cell line), MDA-MB-231 (breast cancer cell line) and KB (cervical cancer cell line). The results show that COMPOUND O exhibited inhibitory effect on the proliferation of these cancer cell lines. The IC50 of COMPOUND O toward these cell lines is similar to those of conventional chemotherapy drugs, paclitaxel and vincristine.

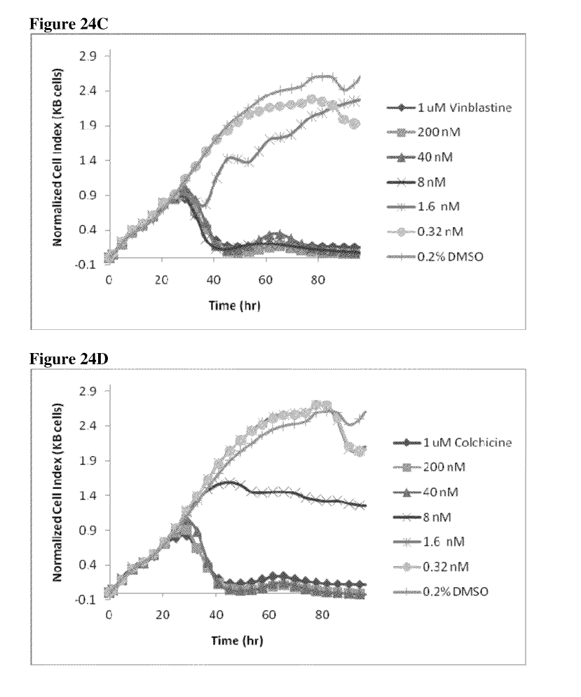

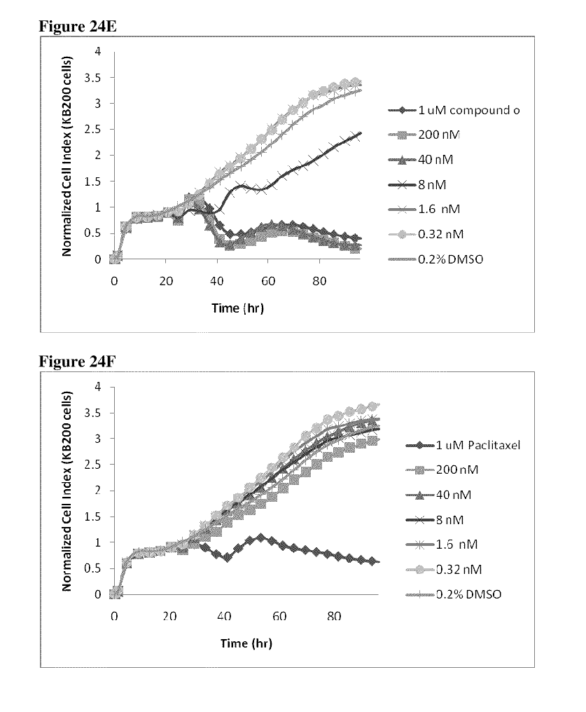

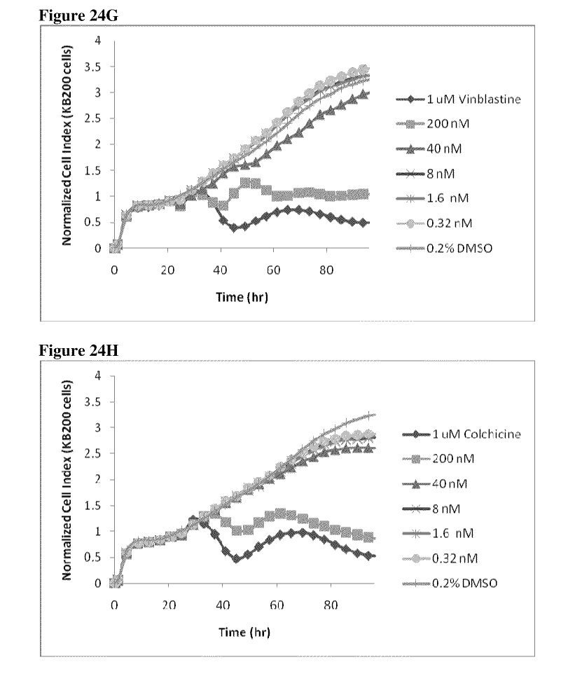

We also tested the effect of COMPOUND O on KB200 over-expressing the multiple drug resistance (MDR) gene. This gene is responsible for resistance of chemotherapy drugs, e.g., paclitaxel and vincristine/vinblastine in clinic. FIGS. 24A-D show that growth of the parental KB cell line was inhibited by COMPOUND O (IC50=33.1 nM), paclitaxel (IC50=7.19 nM), vinblastine (IC50=4.74 nM) and colchicine (IC50=8.20 nM). The IC50 of these 4 compounds are in a similar range. FIG. 24E show that growth of KB200 cells was sensitive to the inhibition by COMPOUND O (IC50=9.84 nM). In contrast, the growth of KB200 cells was much less sensitive to the inhibition by paclitaxel (IC50>1 uM), vinblastine (IC50=135 nM) and colchicine (IC50=116 nM) (as shown in FIGS. 24F-H). This suggests that COMPOUND O can be a great 2^{nd} line therapy toward patients who have failed on paclitaxel and vinblastine treatment.

FIG. 25 shows that non-cancerous cell line NIH-3T3 was much less sensitive to the inhibition by COMPOUND O (IC50=6 uM). Therefore, COMPOUND O demonstrates higher cytotoxicity effect toward cancer cells versus normal cells.

### In Vivo Screening for Anticancer Activity

To evaluate the in vivo anticancer efficacy of the COMPOUND O, three human tumor xenograft models were used. It included MKN45 human gastric-intestinal cancer, H460 human non-small cell lung cancer and A549 human lung cancer xenograft models in immunodeficient nude mice. Details of the in vivo anticancer efficacy of COMPOUND O are provided in Examples 1-3.

### Example 1

### In Vivo Anticancer Activity of COMPOUND O on MKN45 Human Gastric-Intestinal Cancer in Nude Mice

To evaluate the in vivo anticancer efficacy of COMPOUND O, MKN45 human gastric-intestinal cancer xenograft models in immunodeficient nude mice was tested. All the mouse models are maintained in the Pharmacology Lab of ACEA Bio (Hangzhou) CO., Ltd. The BALB/c immunodeficient nude mice were purchased from Shanghai SLAC Laboratory Animal, certification number: SCXKA (Shanghai) 2007-0005. The mouse weight was 19±1 g. Only female mice were used in this study. The numbers of animals tested were as follow: 7 for each dose group, 7 for positive control group and 7 for negative control group.

Test Control. For negative control, each mouse was administered orally with the solvent only having the same volume and same concentration as those used in COMPOUND O test, 120 mg/kg once every other day (qod) for 18 days. For positive control, an oral anticancer compound, Etoposide was administered orally at 50 mg/kg, once every 4 days, for 18 days.

Preparation an Administration of Test Compounds. COMPOUND O was dissolved in 25% phospholipid (S75) and 75% polyvinylpyrrolidone (PL-PVP) then further diluted to 8 mg/ml in 0.9% NaCl aqueous solution. Different dosages of COMPOUND O between 120 mg/kg qod (once every other day) and 160 mg/kg q3d (once every 3 days) were used in the study.

Preparation of tumor cells for transplantation and determination of compound efficacy. To prepare the tumor cells for MKN45 human gastric-intestinal cancer xenograft model, the fast grown tumors were first removed from the transplanted tumor mice, the tumor tissues were dissected to 1-2 mm³ in dimension. These micro-tumors were then subcutaneously injected into the auxiliary region (right-side) of each mouse. After the inoculated tumors grew to a certain size (60-80 mm³) in the nude mice, the mice were randomized into different dosing groups and subjected to compound treatment. Between 2-3 weeks after the first dosing, mice were sacrificed and the transplanted tumors were removed from experimental mice. Each removed solid tumor was weighed; the tumor inhibition rate in each dosage group was calculated according to the formula:

Tumor inhibition rate %=(average weight of tumor in the negative control group−average weight of tumor in the compound treated group)/average weight of tumor in the negative control group×100 (1)

All used materials including animal food, animal cage, supporting materials and apparatus contacted by animals, were high-pressure sterilized. Nude mice were maintained in laminar flow shelves under SPF condition. After tumor transplantation, mouse weight and tumor size in each compound dosage group were dynamically monitored and plotted. The tumor size was determined by measuring the major axis (a) and minor axis (b) of the tumor, and tumor volume was calculated according to the formula:

Tumor volume=a×b²/2 (2)

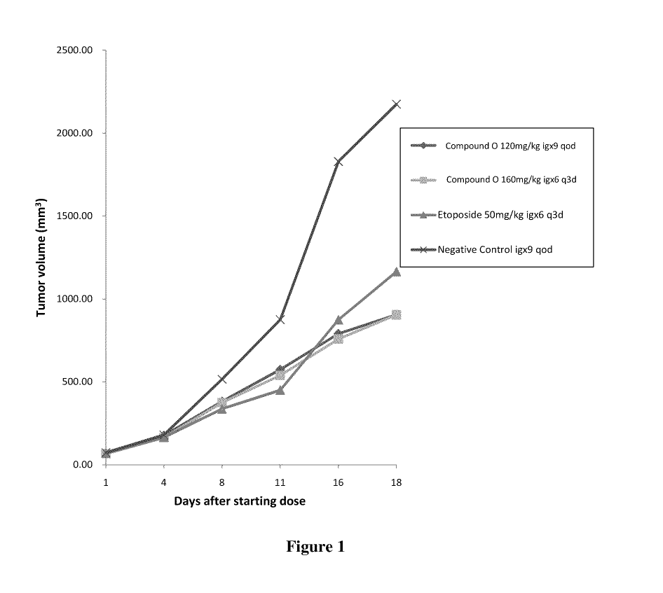

Results. In MKN45 human gastric-intestinal cancer xenograft model in Nude mice, COMPOUND O showed the average in vivo tumor inhibition rate of 58.0% and 55.7%, in 120 mg/kg qod and 160 mg/kg q3d dosage group, respectively. In the same experiment, Etoposide showed an average in vivo tumor inhibition rate of 48.4% for the route administration dosage of 50 mg/kg q3d. The details are provided in Table 2. The dynamic changes of tumor size are summarized in FIG. 1. The dynamic changes of body weight of carrier mice are summarized in Table 3. The anticancer effect of COMPOUND O (120 mg/kg qod) in MKN45 xenograft model is similar to that of Etoposide (30 mg/kg q3d) under the same drug-administration route.

TABLE 2
The in vivo antitumor efficacy of COMPOUND O on MKN45 human Gastric-intestinal Cancer
that was xenograft-transplanted in immunodeficient nude mice by subcutaneous implanting.

| | | | | | | | | | TV |
|---|---|---|---|---|---|---|---|---|---|
| | | | Animal | Animal | | Tumor | | | inhibition |
| | Dosage | Administration | No. | weight (g) | Tumor weight (g) | inhibition | Tumor volume (mm³) | | rate % |
| Sample | (mg/kg) | method | Beginning/end | Beginning/end | x ± SD | rate % | Start | End | T/C |
| COMPOUND O | 120 | ig × 9 qod | 7/7 | 19/16.7 | 0.835 ± 0.41 | 58.0 | 69.58 ± 11 | 905 ± 403 | 44.1 |
| COMPOUND O | 160 | ig × 6 q3d | 7/3 | 18.3/15.7 | 0.881 ± 0.467 | 55.7 | 67.75 ± 25 | 904 ± 347 | 45.3 |
| Etoposide | 50 | ig × 6 q3d | 7/7 | 18.9/16.9 | 1.024 ± 0.345 | 48.4 | 69.46 ± 16 | 1164 ± 483 | 56.9 |
| Negative | solvent | ig × 9 qod | 7/7 | 18.7/22 | 1.987 ± 0.554 | n.a. | 73.76 ± 37 | 2174 ± 702 | n.a. |
| control | | | | | | | | | |

TABLE 3
The dynamic change in body weight of carrier mice in the in vivo antitumor
efficacy test of COMPOUND O on MKN45 human Gastric-intestinal Cancer that was
xenograft-transplanted in immunodeficient nude mice by subcutaneous implanting.

| | Dosage | Administration | Body weight of mice (g) | | | | | |
|---|---|---|---|---|---|---|---|---|
| Sample | (mg/kg) | method | 1 day | 4 day | 8 day | 11 day | 16 day | 18 day |
| COMPOUND O | 120 | ig × 9 qod | 19.03 ± 0.76 | 19.53 ± 0.71 | 18.24 ± 1.10 | 18.21 ± 1.02 | 17.09 ± 1.09 | 16.71 ± 0.97 |
| COMPOUND O | 160 | ig × 6 q3d | 18.37 ± 0.66 | 19.10 ± 0.73 | 17.33 ± 1.52 | 17.15 ± 1.33 | 16.43 ± 1.47 | 15.73 ± 1.31 |
| Etoposide | 50 | ig × 6 q3d | 18.94 ± 0.92 | 19.57 ± 1.16 | 17.17 ± 0.85 | 17.26 ± 1.24 | 16.94 ± 0.91 | 16.93 ± 0.75 |
| Negative control | solvent | ig × 9 qod | 18.79 ± 0.91 | 19.18 ± 0.50 | 19.77 ± 0.70 | 20.95 ± 0.88 | 20.43 ± 0.94 | 20.22 ± 1.00 |

### Example 2

### In Vivo Anticancer Activity of COMPOUND O on H460 Human Non-Small Cell Lung Cancer in Nude Mice

To evaluate the in vivo anticancer efficacy of COMPOUND O, H460 human non-small cell lung Cancer xenograft models in immunodeficient nude mice was used. The cell line and mice were maintained in the Pharmacology Lab of ACEA Bio (Hangzhou) CO., Ltd. The BALB/c immunodeficient nude mice were purchased from Shanghai SLAC Laboratory Animal, certification number: SCXKA (Shanghai) 2007-0005. The mouse weight was between 19±2 g. Only female mice were used in this study. The numbers of animals tested were as follows: 7 for each dose group, 7 for positive control group and 7 for negative control group.

Test Control. For negative control, each mouse was administered orally with the solvent only having the same volume and same concentration as those used in COMPOUND O test, 120 mg/kg once every other day (qod) for 25 days. For positive control, an oral anticancer compound, Etoposide was administered orally at 30 mg/kg qod for 25 days.

Preparation an Administration of Test Compounds. COMPOUND O was dissolved in 25% phospholipid (S75) and 75% polyvinylpyrrolidone (PL-PVP) then further diluted to 8 mg/ml in 0.9% NaCl aqueous solution. Each mouse was administered orally with the compound solution. Different dosages of COMPOUND O (120 mg/kg qod and 160 mg/kg q3d) were used in the study.

Preparation of tumor cells for transplantation and determination of compound efficacy. To prepare the cancer cells for H460 human non-small cell lung cancer xenograft model, the log phase growing cell in the flask were trypsinized and cells were re-suspended in PBS (pH 7.2) at 1.5×10⁷ cells/ml. The cell suspension (3×10⁶ cells) was subcutaneously injected into the auxiliary region (right-side) of each mouse. After the inoculated cancer cells grew to a tumor of certain size (150 mm³) in the nude mice, the mice were randomized into different dosing groups and subjected to compound treatment. Between 3-4 weeks after the first dosing, mice were sacrificed and the transplanted tumors were removed from experimental mice. Each removed solid tumor was weighed; the tumor inhibition rate in each dosage group was calculated according to equation (1) in Example 1.

All used materials including animal food, animal cage, supporting materials and apparatus contacted by animals, were high-pressure sterilized. Nude mice were maintained in laminar flow shelves under SPF condition. After tumor transplantation, mouse weight and tumor size in each compound dosage group were dynamically monitored and plotted. The tumor size was determined by measuring the major axis (a) and minor axis (b) of the tumor, and tumor volume was calculated according to the formula (2) in Example 1.

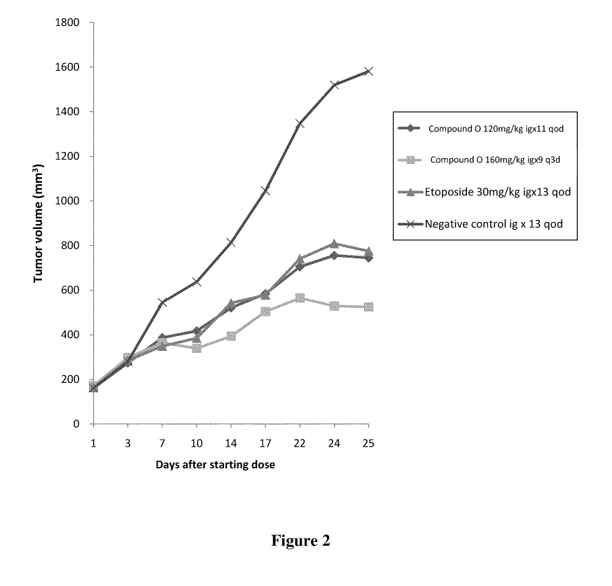

Results. In H460 human non-small cell lung cancer xenograft model in Nude mice, COMPOUND O showed the average in vivo tumor inhibition rate of 47.1% and 31.3%, in 120 mg/kg qod and 160 mg/kg q3d dosage group, respectively. In the same experiment, Etoposide showed an average in vivo tumor inhibition rate of 48.4% for the route administration dosage of 30 mg/kg qod. The details are provided in Table 4. The dynamic changes of tumor size are summarized in FIG. 2. The dynamic changes of body weight of carrier mice are summarized in Table 5. The anticancer effect of COMPOUND O (120 mg/kg) in H460 human non-small cell lung cancer xenograft model is similar to that of Etoposide (30 mg/kg) under the same drug-administration procedure.

TABLE 4
The in vivo antitumor efficacy of COMPOUND O on H460 human Non-small cell lung
Cancer that was xenograft-transplanted in immunodeficient nude mice by subcutaneous implanting.

| | | | | Animal | | | | | TV |
|---|---|---|---|---|---|---|---|---|---|
| | | Admin- | Animal No. | weight (g) | | Tumor | | | inhibition |
| | Dosage | istration | Beginning/ | Beginning/ | Tumor weight (g) | inhibition | Tumor volume (mm³) | | rate % |
| sample | (mg/kg) | method | end | end | x ± SD | rate % | Start | End | T/C |
| COMPOUND | 120 | ig × 11 qod | 7/7 | 20.04/17.33 | 0.81 ± 0.29* | 47.1 | 162.09 ± 53.94 | 745.28 ± 245.55 | 47.0 |
| O | | | | | | | | | |
| COMPOUND | 160 | ig × 9 q3d | 7/7 | 20.00/16.24 | 0.46 ± 0.13** | 31.3 | 171.44 ± 53.63 | 524.67 ± 141.77 | 69.9 |
| O | | | | | | | | | |
| Etoposide | 30 | ig × 13 qod | 7/7 | 19.97/17.13 | 0.76 ± 0.29* | 48.4 | 163.79 ± 57.22 | 775.43 ± 302.62 | 50.4 |
| Negative | solvent | ig × 13 qod | 7/7 | 20.02/22.69 | 1.53 ± 0.76 | n.a. | 161.80 ± 33.35 | 1581.09 ± 553.8 | n.a. |
| control | | | | | | | | | |

TABLE 5
The dynamic change in body weight of carrier mice in the in vivo antitumor efficacy
test of COMPOUND O on H460 human Non-small cell lung Cancer that was xenograft-
transplanted in immunodeficient nude mice by subcutaneous implanting.

| | Dosage | Administration | Body weight of mice (g) | | | | |
|---|---|---|---|---|---|---|---|
| sample | (mg/kg) | method | 1 day | 3 day | 7 day | 10 day | 14 day |
| COMPOUND O | 120 | ig × 11 qod | 20.04 ± 0.51 | 19.80 ± 1.18 | 18.86 ± 0.70 | 18.20 ± 0.68 | 18.11 ± 0.49 |
| COMPOUND O | 160 | ig × 9 q3d | 20.00 ± 0.48 | 20.54 ± 0.53 | 18.64 ± 0.61 | 17.89 ± 0.39 | 17.83 ± 0.57 |
| Etoposide | 30 | ig × 13 qod | 19.97 ± 0.88 | 19.66 ± 0.75 | 19.70 ± 1.38 | 19.66 ± 1.10 | 18.86 ± 1.07 |
| Negative control | solvent | ig × 13 qod | 20.04 ± 0.62 | 20.49 ± 0.72 | 21.11 ± 0.60 | 21.39 ± 0.56 | 21.86 ± 0.67 |

| | Dosage | Administration | Body weight of mice (g) | | | |
|---|---|---|---|---|---|---|
| sample | (mg/kg) | method | 17 day | 22 day | 24 day | 25 day |
| COMPOUND O | 120 | ig × 11 qod | 17.43 ± 0.51 | 17.24 ± 0.72 | 17.56 ± 1.21 | 17.33 ± 1.23 |
| COMPOUND O | 160 | ig × 9 q3d | 18.24 ± 0.46 | 16.91 ± 0.97 | 16.53 ± 1.29 | 16.24 ± 1.55 |
| Etoposide | 30 | ig × 13 qod | 18.79 ± 1.17 | 17.86 ± 1.35 | 17.56 ± 1.44 | 17.13 ± 1.14 |
| Negative control | solvent | ig × 13 qod | 21.69 ± 0.94 | 22.44 ± 1.06 | 22.69 ± 1.03 | 22.46 ± 0.85 |

### Example 3

### In Vivo Anticancer Activity of COMPOUND O on A549 Human Non-Small Cell Lung Cancer in Nude Mice

To evaluate the in vivo anticancer efficacy of COMPOUND O, A549 human non-small cell lung cancer xenograft models in immunodeficient nude mice was used. The cell line and mice were maintained in the Pharmacology Lab of ACEA Bio (Hangzhou) CO., Ltd. The BALB/c immunodeficient nude mice were purchased from Shanghai SLAC Laboratory Animal, certification number: SCXKA (Shanghai) 2007-0005. The mouse weight was between 21±1 g. Only female mice were used in this study. The numbers of animals tested were as follow: 7-8 for each dose group, 6 for positive control group and 7 for negative control group.

Test Control. For negative control, each mouse was administered orally with the solvent only having the same volume and same concentration as those used in COMPOUND O test, 120 mg/kg once every other day (qod) for 25 days. For positive control, an oral anticancer compound, Etoposide was administered orally at 30 mg/kg qod for 25 days.

Preparation an Administration of Test Compounds. COMPOUND O was dissolved in 25% phospholipid (S75) and 75% polyvinylpyrrolidone (PL-PVP) then further diluted to 8 mg/ml in 0.9% NaCl aqueous solution. Each mouse was administered orally with the compound solution. Different dosages of COMPOUND O (120 mg/kg qod and 160 mg/kg q2*2 (dosing for 2 consecutive days then off for 2 consecutive days) were used in the study.

Preparation of tumor cells for transplantation and determination of compound efficacy. To prepare the cancer cells for A549 human non-small cell lung cancer xenograft model, the log phase growing cell in the flask were trypsinized and cells were re-suspended in PBS (pH 7.2) at 2.5×10⁷ cells/ml. The cell suspension (5×10⁶ cells) was subcutaneously injected into the auxiliary region (right-side) of each mouse. After the inoculated cancer cells grew to a tumor of certain size (50-60 mm³) in the nude mice (about 15 days post inoculation), the mice were randomized into different dosing groups and subjected to compound treatment. Between 3-4 weeks after the first dosing, mice were sacrificed and the transplanted tumors were removed from experimental mice. Each removed solid tumor was weighed; the tumor inhibition rate in each dosage group was calculated according to equation (1) in Example 1.

All used materials including animal food, animal cage, supporting materials and apparatus contacted by animals, were high-pressure sterilized. Nude mice were maintained in laminar flow shelves under SPF condition. After tumor transplantation, mouse weight and tumor size in each compound dosage group were dynamically monitored and plotted. The tumor size was determined by measuring the major axis (a) and minor axis (b) of the tumor, and tumor volume was calculated according to the formula (2) in Example 1.

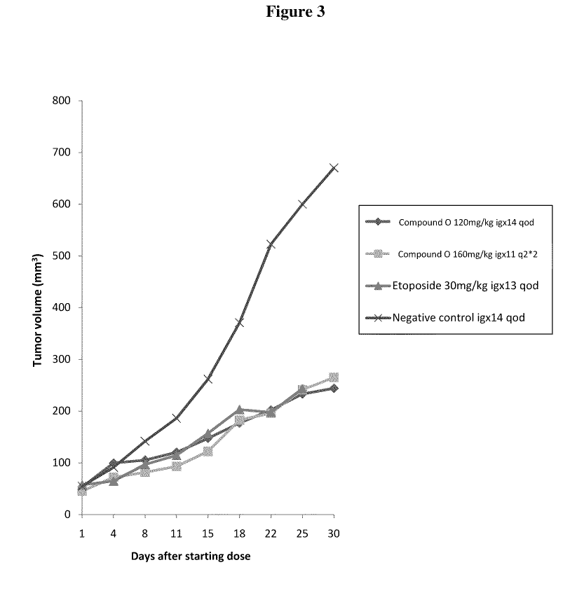

Results. In A549 human non-small cell lung cancer xenograft model in nude mice, COMPOUND O showed the average in vivo tumor inhibition rate of 67.8% and 62.5% in 120 mg/kg qod and 160 mg/kg q2*2 dosage group, respectively. In the same experiment, Etoposide showed an average in vivo tumor inhibition rate of 59.9% for the route administration dosage of 30 mg/kg qod. The details are provided in Table 6. The dynamic changes of tumor size are summarized in FIG. 3. The dynamic changes of body weight of carrier mice are summarized in Table 7. The anticancer effect of COMPOUND O (120 mg/kg) in A549 human non-small cell lung Cancer xenograft model is similar to that of Etoposide (30 mg/kg) under the same drug-administration procedure.

TABLE 6
The in vivo antitumor efficacy of COMPOUND O on A549 human Non-small cell lung
Cancer that was xenograft-transplanted in immunodeficient nude mice by subcutaneous
implanting.

| | | | | | | | | | TV |
|---|---|---|---|---|---|---|---|---|---|
| | | | | Animal | Tumor | | | | inhibition |
| | Dosage | Administration | Animal No. | weight (g) | weight (g) | Tumor | Tumor volume (mm³) | | rate % |
| sample | (mg/kg) | method | Beginning/end | Beginning/end | x ± SD | inhibition rate % | Start | End | T/C |
| COMPOUND O | 120 | ig × 14 qod | 7/7 | 20.77/16.94 | 0.25 ± 0.08 | 67.8 | 53.31 ± 24.5 | 244 ± 105 | 37.5 |
| COMPOUND O | 160 | ig × 11 q2*2 | 8/3 | 21.69/17.97 | 0.29 ± 0.11 | 62.5 | 45.25 ± 15.9 | 265 ± 135 | 47.9 |
| Etoposide | 30 | ig × 13 qod | 6/6 | 21.30/18.18 | 0.27 ± 0.15 | 59.9 | 57.42 ± 23.5 | 243 ± 121 | 40.0 |
| Negative | solvent | ig × 14 qod | 7/7 | 21.15/24/20 | 0.76 ± 0.22 | n.a. | 54.83 ± 12.4 | 670 ± 202 | n.a. |
| control | | | | | | | | | |

TABLE 7
The dynamic change in body weight of carrier mice in the in vivo antitumor efficacy test of
COMPOUND O on A549 human Non-small cell lung Cancer that was xenograft-transplanted in
immunodeficient nude mice by subcutaneous implanting.

| | | Admin- | | | | | | | | |
|---|---|---|---|---|---|---|---|---|---|---|
| | Dosage | istration | Body weight of mice (g) | | | | | | | |
| sample | (mg/kg) | method | 1 day | 4 day | 8 day | 11 day | 15 day | 18 day | 22 day | 25 day |
| COM- | 120 | ig × 14 qod | 20.77 ± 0.68 | 20.27 ± 0.83 | 19.57 ± 1.09 | 18.20 ± 0.59 | 17.90 ± 0.61 | 17.90 ± 1.09 | 17.21 ± 1.20 | 17.00 ± 1.29 |
| POUND | | | | | | | | | | |
| O | | | | | | | | | | |
| COM- | 160 | ig × 11 q2*2 | 21.69 ± 0.66 | 21.20 ± 0.79 | 19.00 ± 1.48 | 17.78 ± 2.11 | 17.43 ± 1.86 | 18.43 ± 0.90 | 18.70 ± 1.04 | 17.97 ± 0.93 |
| POUND | | | | | | | | | | |
| O | | | | | | | | | | |
| Etoposide | 30 | ig × 13 qod | 21.30 ± 0.70 | 19.38 ± 2.22 | 20.53 ± 1.26 | 19.62 ± 1.15 | 19.63 ± 1.63 | 19.48 ± 2.01 | 19.10 ± 2.57 | 18.18 ± 1.86 |
| Negative | solvent | ig × 14 qod | 21.15 ± 0.67 | 21.54 ± 0.55 | 22.45 ± 0.52 | 22.54 ± 0.56 | 23.40 ± 0.58 | 23.43 ± 0.57 | 23.73 ± 0.62 | 24.24 ± 0.55 |
| control | | | | | | | | | | |

### Example 4

### Inhibition of Cell Proliferation by COMPOUND O, Paclitaxel and Vincristine in A549 Cells

A549 cells (human lung carcinoma cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using RT-CES system (ACEA Biosciences). The RT-CES system is the same as the xCelligence system, currently available from Roche). FIGS. 4A-C show the normalized cell index as a function of time prior to and after the compound addition. The cell index was normalized against the cell index values at a time point just before compound addition. The calculated IC50 (72 hr post compound treatment) is 10.9 nM, 5.3 nM and 61.8 nM for COMPOUND O, paclitaxel and vincristine, respectively.

### Example 5

### Inhibition of Cell Proliferation by COMPOUND O in H596 Cells

H596 cells (human adenosquamous lung carcinoma cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). The xCelligence system is the same as the RT-CES system from ACEA Biosciences. FIG. 5 shows the normalized cell index as a function of time prior to and after COMPOUND O addition. The Cell index was normalized against the cell index values at a time point just before compound addition. The calculated IC50 (72 hr post compound treatment) is 37.9 nM for COMPOUND O. In comparison, the calculated IC50 (72 hr post compound treatment) are 12.5 nM and 36.9 nM for paclitaxel and vincristine, respectively.

### Example 6

### Inhibition of Cell Proliferation by COMPOUND O in H292 Cells

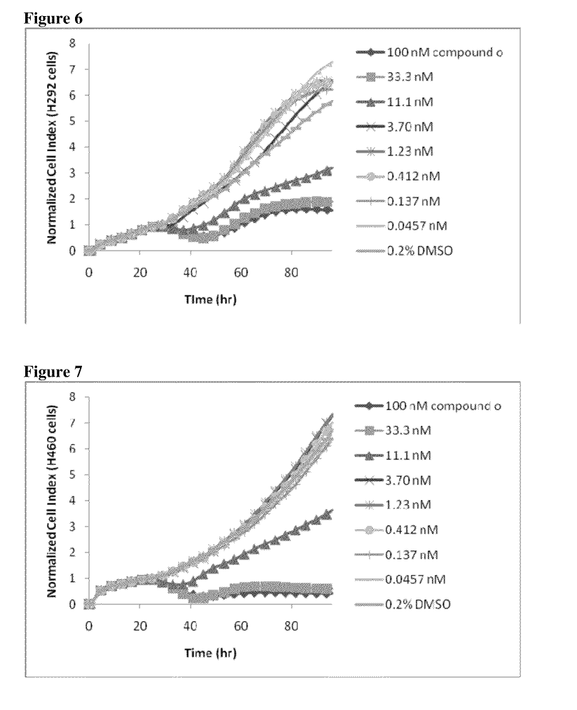

H292 cells (human pulmonary lung carcinoma cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 6 shows the normalized cell index as a function of time prior to and after COMPOUND O addition. The Cell index was normalized against the cell index values at a time point just before compound addition. The calculated IC50 (72 hr post compound treatment) is 10.8 nM for COMPOUND O. In comparison, the calculated IC50 (72 hr post compound treatment) are 2.59 nM and 2.63 nM for paclitaxel and vincristine, respectively.

### Example 7

### Inhibition of Cell Proliferation by COMPOUND O in H460 Cells

NCI-H460 cells (human large cell lung carcinoma cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 7 shows the normalized cell index as a function of time prior to and after COMPOUND O addition. The Cell index was normalized against the cell index values at a time point just before compound addition. The calculated IC50 (72 hr post compound treatment) is 10.9 nM for COMPOUND O. In comparison, the calculated IC50 (72 hr post compound treatment) are 2.68 nM and 10.4 nM for paclitaxel and vincristine, respectively.

### Example 8

### Inhibition of Cell Proliferation by COMPOUND O in H1993 Cells

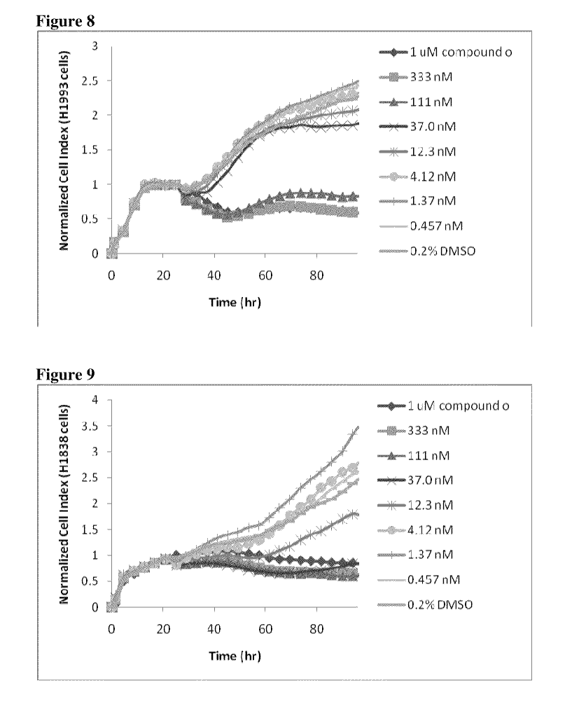

H1993 cells (human non-small cell lung cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 8 shows the normalized cell index as a function of time prior to and after COMPOUND O addition. The Cell index was normalized against the cell index values at a time point just before compound addition. The calculated IC50 (72 hr post compound treatment) is 60.2 nM for COMPOUND O. In comparison, the calculated IC50 (72 hr post compound treatment) are 5.12 nM and 2.43 nM for paclitaxel and vincristine, respectively.

### Example 9

### Inhibition of Cell Proliferation by COMPOUND O in H1838 Cells

H1838 cells (human non-small cell lung cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 9 shows the normalized cell index as a function of time prior to and after COMPOUND O addition. The Cell index was normalized against the cell index values at a time point just before compound addition. The calculated IC50 (72 hr post compound treatment) is 11.8 nM for COMPOUND O. In comparison, the calculated IC50 (72 hr post compound treatment) is 3.16 nM for vincristine.

### Example 10

### Inhibition of Cell Proliferation by COMPOUND O in H2347 Cells

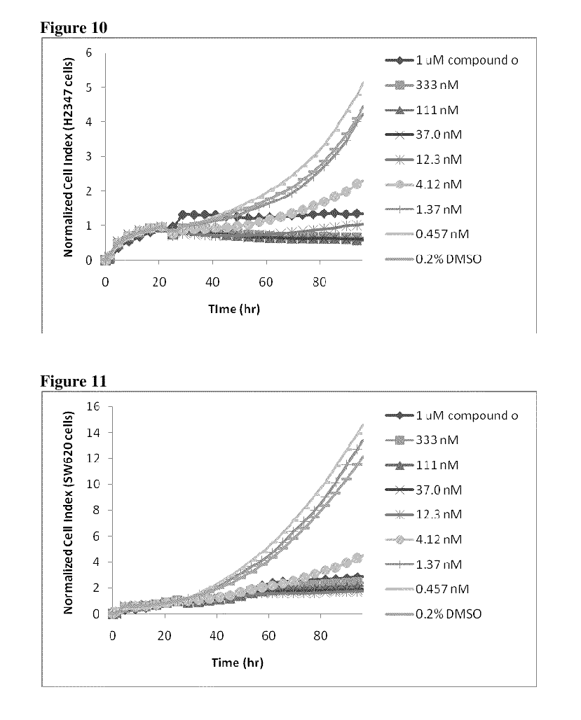

H2347 cells (human non-small cell lung cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 10 shows the normalized cell index as a function of time prior to and after COMPOUND O addition. The Cell index was normalized against the cell index values at a time point just before compound addition. The calculated IC50 (72 hr post compound treatment) is 1.76 nM for COMPOUND O. In comparison, the calculated IC50 (72 hr post compound treatment) is 5.05 nM for vincristine.

### Example 11

### Inhibition of Cell Proliferation by COMPOUND O in SW620 Cells

SW620 cells (human colon cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 11 shows the normalized cell index as a function of time prior to and after COMPOUND O addition. The Cell index was normalized against the cell index values at a time point just before compound addition. The calculated IC50 (72 hr post compound treatment) is 2.65 nM for COMPOUND O. In comparison, the calculated IC50 (72 hr post compound treatment) is 0.998 nM for paclitaxel.

### Example 12

### Inhibition of Cell Proliferation by COMPOUND O in GTL16 Cells

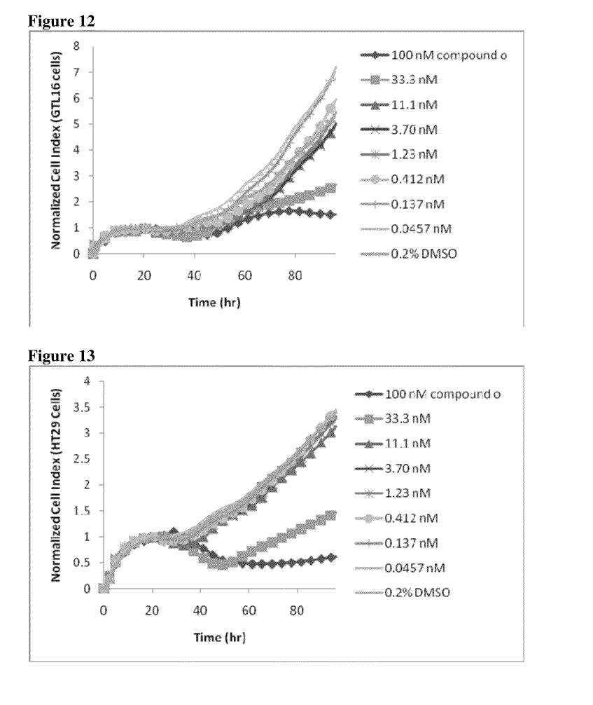

GTL16 cells (human gastric cancer cell line which was derived from MKN45 human gastric cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 12 shows the normalized cell index as a function of time prior to and after COMPOUND O addition. The Cell index was normalized against the cell index values at a time point just before compound addition. The calculated IC50 (72 hr post compound treatment) is 67.0 nM for COMPOUND O. In comparison, the calculated IC50 (72 hr post compound treatment) are 1.02 nM and 3.75 nM for paclitaxel and vincristine, respectively.

### Example 13

### Inhibition of Cell Proliferation by COMPOUND O in HT29 Cells

HT29 cells (human colon cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 13 shows the normalized cell index as a function of time prior to and after COMPOUND O addition. The Cell index was normalized against the cell index values at a time point just before compound addition. The calculated IC50 (72 hr post compound treatment) is 25.4 nM for COMPOUND O. In comparison, the calculated IC50 (72 hr post compound treatment) are 2.52 nM and 12.7 nM for paclitaxel and vincristine, respectively.

### Example 14

### Inhibition of Cell Proliferation by COMPOUND O in A172 Cells

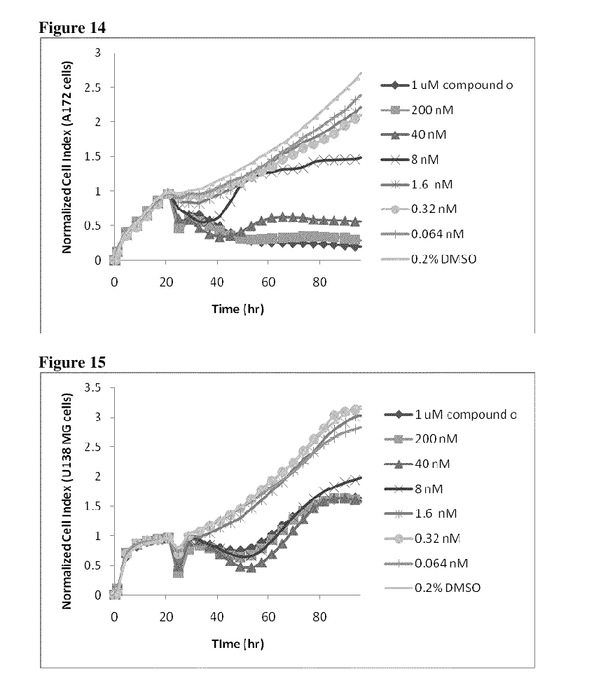

A172 cells (human brain cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 14 shows the normalized cell index as a function of time prior to and after COMPOUND O addition. The Cell index was normalized against the cell index values at a time point just before compound addition. The calculated IC50 (72 hr post compound treatment) is 11.6 nM, 4.46 nM and 1.30 nM for COMPOUND O, paclitaxel and vincristine, respectively.

### Example 15

### Inhibition of Cell Proliferation by COMPOUND O in U138MG Cells

U138MG cells (human brain cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 15 shows the normalized cell index as a function of time prior to and after COMPOUND O addition. The Cell index was normalized against the cell index values at a time point just before compound addition. The calculated IC50 (72 hr post compound treatment) is 3.41 nM, 18.1 nM and 0.641 nM for COMPOUND O, paclitaxel and vincristine, respectively.

### Example 16

### Inhibition of Cell Proliferation by COMPOUND O in U118MG Cells

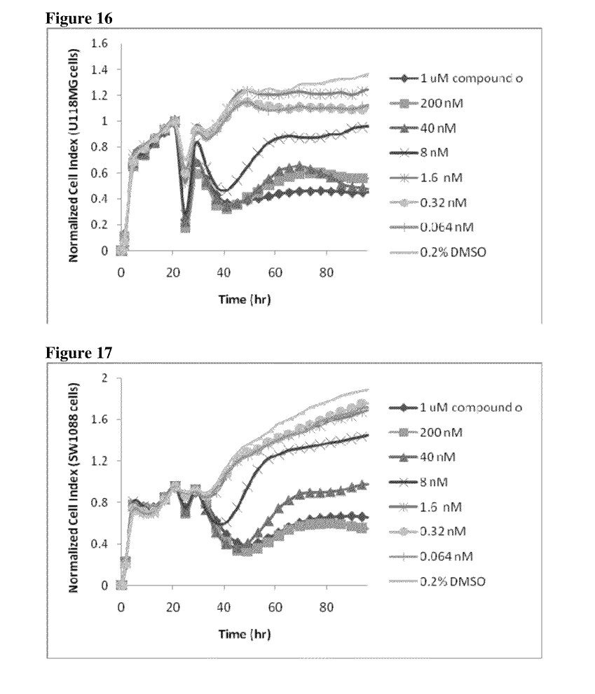

U118MG cells (human brain cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 16 shows the normalized cell index as a function of time prior to and after COMPOUND O addition. The Cell index was normalized against the cell index values at a time point just before compound addition. The calculated IC50 (72 hr post compound treatment) is 13.1 nM, 12.3 nM and 4.31 nM for COMPOUND O, paclitaxel and vincristine, respectively.

### Example 17

### Inhibition of Cell Proliferation by COMPOUND O in SW1088 Cells

SW1088 cells (human brain cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 17 shows the normalized cell index as a function of time prior to and after COMPOUND O addition. The Cell index was normalized against the cell index values at a time point just before compound addition. The calculated IC50 (72 hr post compound treatment) is 21.2 nM, 13.1 nM and 4.96 nM for COMPOUND O, paclitaxel and vincristine, respectively.

### Example 18

### Inhibition of Cell Proliferation by COMPOUND O in HT1080 Cells

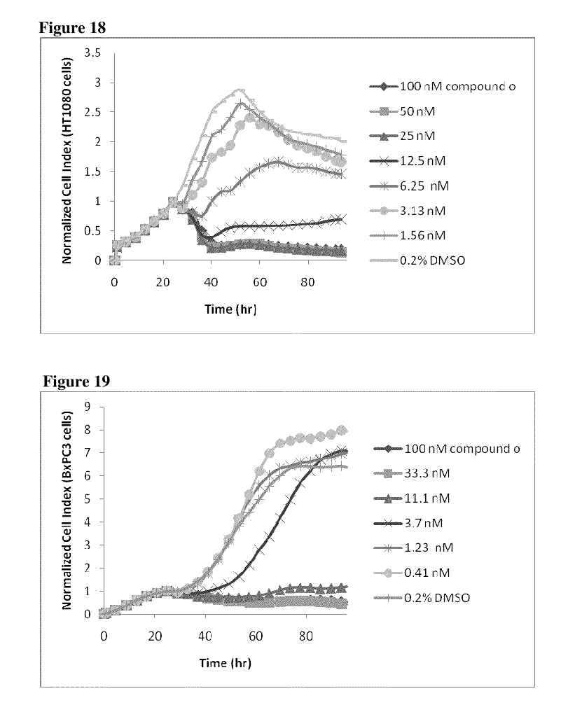

HT1080 cells (human connective tissue cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 18 shows the normalized cell index as a function of time prior to and after COMPOUND O addition. The Cell index was normalized against the cell index values at a time point just before compound addition. The calculated IC50 (72 hr post compound treatment) is 7.63 nM and 2.01 nM for COMPOUND O and vincristine, respectively.

### Example 19

### Inhibition of Cell Proliferation by COMPOUND O in B×PC3 Cells

B×PC3 cells (human pancreatic cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 19 shows the normalized cell index as a function of time prior to and after COMPOUND O addition. The Cell index was normalized against the cell index values at a time point just before compound addition. The calculated IC50 (72 hr post compound treatment) is 6.60 nM for COMPOUND O. In comparison, the calculated IC50 (72 hr post compound treatment) are 8.86 nM and 4.06 nM for paclitaxel and vincristine, respectively.

### Example 20

### Inhibition of Cell Proliferation by COMPOUND O in HepG2 Cells

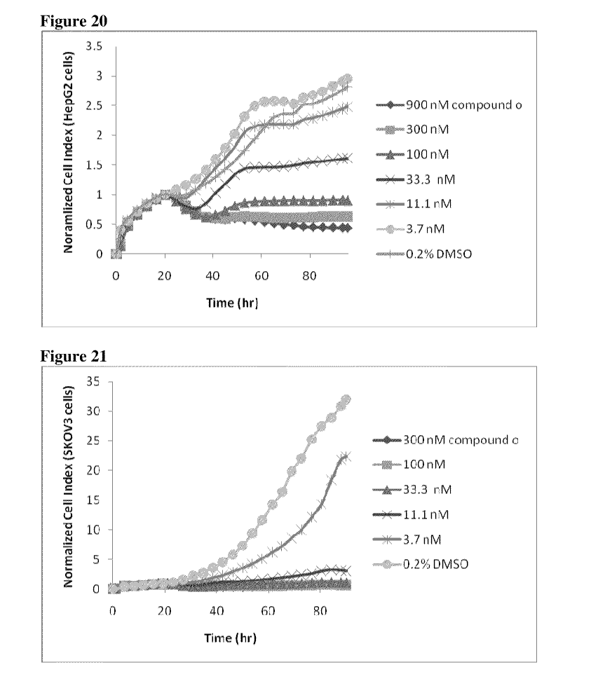

HepG2 cells (human liver cancer cell line) were seeded into wells of 96 well E-plate devices (Roche) with an initial seeding density of 10,000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 20 shows the normalized cell index as a function of time prior to and after COMPOUND O addition. The Cell index was normalized against the cell index values at a time point just before compound addition. The calculated IC50 (72 hr post compound treatment) is 25.9 nM for COMPOUND O.

### Example 21

### Inhibition of Cell Proliferation by COMPOUND O in SKOV3 Cells

SKOV3 cells (human ovarian cancer cell line) were seeded into wells of 96 well E-plate devices (Roche) with an initial seeding density of 3000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 21 shows the normalized cell index as a function of time prior to and after COMPOUND O addition. The Cell index was normalized against the cell index values at a time point just before compound addition. The calculated IC50 (72 hr post compound treatment) is 5.47 nM for COMPOUND O. In comparison, the calculated IC50 (72 hr post compound treatment) are 5.34 nM for paclitaxel.

### Example 22

### Inhibition of Cell Proliferation by COMPOUND O in MCF7 Cells

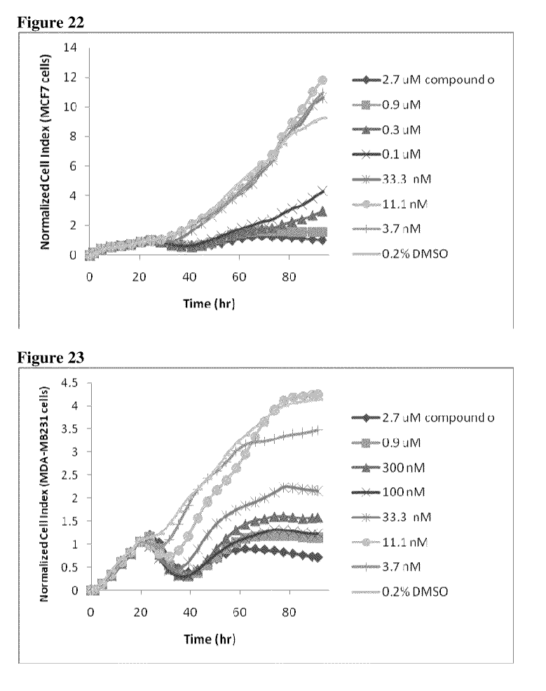

MCF7 cells (human breast cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 22 shows the normalized cell index as a function of time prior to and after COMPOUND O addition. The Cell index was normalized against the cell index values at a time point just before compound addition. The calculated IC50 (72 hr post compound treatment) is 74.4 nM for COMPOUND O. In comparison, the calculated IC50 (72 hr post compound treatment) are 11.3 nM paclitaxel.

### Example 23

### Inhibition of Cell Proliferation by COMPOUND O in MDA-MB-231 Cells

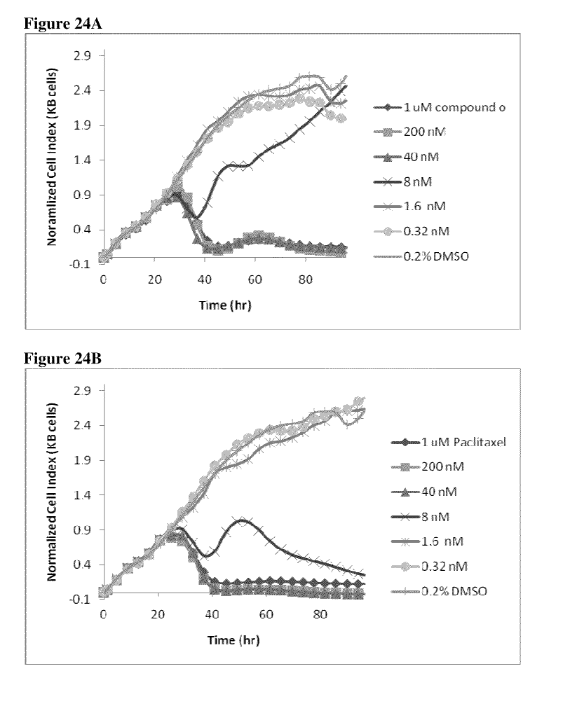

MDA-MB-231 cells (human breast cancer cell line) were seeded into wells of 96 well E-plate devices (Roche) with an initial seeding density of 5000 cells per well and were pre-incubated in incubator under standard cell culture condition for about 24 hours. COMPOUND O, paclitaxel and vincristine at different concentrations in DMSO were added into wells following the incubation period. The cell status was monitored prior to and after the compound addition using xCelligence system (Roche). FIG. 23 shows the normalized cell index as a function of time prior to and after COMPOUND O addition. The Cell index was normalized against the cell index values at a time point just before compound addition. The calculated IC50 (72 hr post compound treatment) is 32.4 nM for COMPOUND O.

### Example 24

### Inhibition of Cell Proliferation by COMPOUND O in KB200 Cells Over-Expressing the Multiple Drug Resistant (MDR) Gene

The parental cervical cancer cell line, KB cells were first tested. KB cells were seeded into wells of 96 well E-plate devices (Roche) with an initial seeding density of 5000 cells per well and were pre-incubated in incubator under standard cell culture condition for about 24 hours. COMPOUND O, paclitaxel, vinblastine and colchicine at different concentrations in DMSO were added into wells following the incubation period. The cell status was monitored prior to and after the compound addition using xCelligence system (Roche). FIGS. 24A-D show the normalized cell index as a function of time prior to and after the compound addition. The Cell index was normalized against the cell index values at a time point just before compound addition. The calculated IC50 (72 hr post compound treatment) is 33.1 nM, 7.19 nM, 4.74 nM and 8.20 nM for COMPOUND O, paclitaxel, vinblastine and colchicine, respectively.

The efficacy of these compounds was also tested against KB200 cell over-expressing multiple drug resistant (MDR) gene. Similarly, KB200 cells were seeded into wells of 96 well E-plate devices (Roche) with an initial seeding density of 5000 cells per well and were pre-incubated in incubator under standard cell culture condition for about 24 hours. COMPOUND O, paclitaxel, vinblastine and colchicine at different concentrations in DMSO were added into wells following the incubation period. The cell status was monitored prior to and after the compound addition using xCelligence system (Roche). FIGS. 24E-H show the normalized cell index as a function of time prior to and after the compound addition. The Cell index was normalized against the cell index values at a time point just before compound addition. The calculated IC50 (72 hr post compound treatment) is 9.84 nM, 0.135 μM and 0.116 μM for COMPOUND O, vinblastine and colchicine, respectively. The calculated IC50 (72 hr post compound treatment) for paclitaxel is >1 μM. COMPOUND O shows good efficacy toward cell line that is resistant to conventional chemotherapy (e.g., paclitaxel and vinblastine). This suggests that COMPOUND O can be a great 2^{nd} line therapy toward patients who have failed on paclitaxel and vinblastine treatment.

### Example 25

### Inhibition of Cell Proliferation by COMPOUND O in NIH 3T3 Normal Tissue Cell Line

NIH 3T3 cells (the normal tissue cell line) were seeded into wells of 96 well E-plate devices (Roche) with an initial seeding density of 5000 cells per well and were pre-incubated in incubator under standard cell culture condition for about 24 hours. COMPOUND O at different concentrations in DMSO were added into wells following the incubation period. The cell status was monitored prior to and after the compound addition using xCelligence system (Roche). FIG. 25 shows the normalized cell index as a function of time prior to and after COMPOUND O addition. The Cell index was normalized against the cell index values at a time point just before compound addition. The calculated IC50 (72 hr post compound treatment) is 6.0 μM for COMPOUND O. In contrast, the IC50 of COMPOUND O towards all tested cancer cell lines is in low nM range. COMPOUND O demonstrates higher cytotoxicity effect toward cancer cells versus normal cells.

### Example 26

### Inhibition of Tubulin Polymerization In vitro and In vivo by COMPOUND O

Microtubules are important in numerous cellular processes, including mitosis when the duplicated chromosomes are separated into two identical sets before cleavage of the cell into two daughter cells. The key role of microtubules and their dynamics in mitosis and cell division make microtubules an important target for anticancer drugs. In cells during interphase, microtubules exchange their tubulin with soluble tubulin in the cytoplasmic pool with half times of ˜3 minutes to several hours. With the onset of mitosis, the interphase microtubule network disassembles and is replaced by population of highly dynamic microtubules which forms the mitotic spindle and moves the chromosomes. Mitotic spindle microtubules are 20-50 times more dynamic than microtubules in interphase cells, and more spindle microtubules exchange their tubulin with tubulin in the soluble pool with half-times as rapid as 15 seconds.

The dynamics of mitotic spindle microtubules are exquisitely sensitive to modulation by regulators and to disruption by microtubule-active drugs. Microtublule-targeted drugs can alter microtubule polymerization and dynamics in a wide variety of ways. Here we demonstrate the mechanisms of action of COMPOUND O: (1) it inhibits microtubule assembly in vitro, (2) it influence microtubule network in cultured cells and (3) it inhibits tubulin assembly via a similar mechanism as Colchicine.

Methods

In vitro microtubule assembly assay. Microtubule polymerization was conducted in a 96-well microtiter plate with MAP-rich tubulin and various concentrations of COMPOUND O in a buffer containing 80 mM PIPES pH 6.9, 0.5 mM EGTA, 2 mM MgCl₂, 1 mM GTP, 10% glycerol and 4% (v/v) dimethyl sulfoxide (DMSO) based on the HTS-tubulin polymerization assay kit protocol (Cytoskeleton). The increase in absorbance was measured at 405 nm a Beckman Multimode DTX880 plate reader at 37° C. and recorded every 60 seconds for 30 minutes.

Spin column assay. Tubulin was incubated with [[³H]vinblastine or [³H]colchicine in the presence of different concentrations of either unlabeled vinblastine, colchicine or COMPOUND O in a buffer containing 0.05 M PIPES, pH 6.9, 1 mM MgCl₂, and 1 mM GTP. The reaction mixtures were incubated at 37° C. for 1 h. The samples were loaded onto Illustra™ MicroSpin™ G-50 columns (GE Healthcare) previously equilibrated with the buffer solution. The columns were placed into 1.5-ml tubes and spun at 750 g for 2 min at room temperature, and radioactivity in the flow-through was analyzed by a scintillation counter.

Results

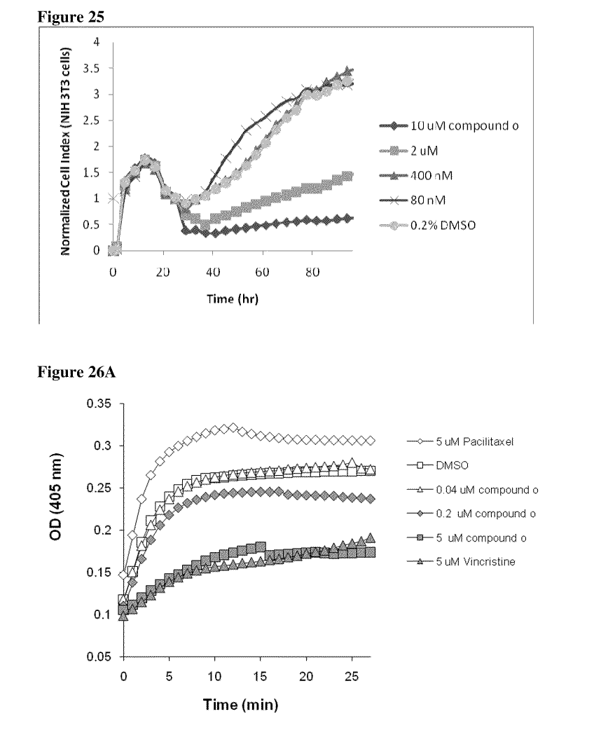

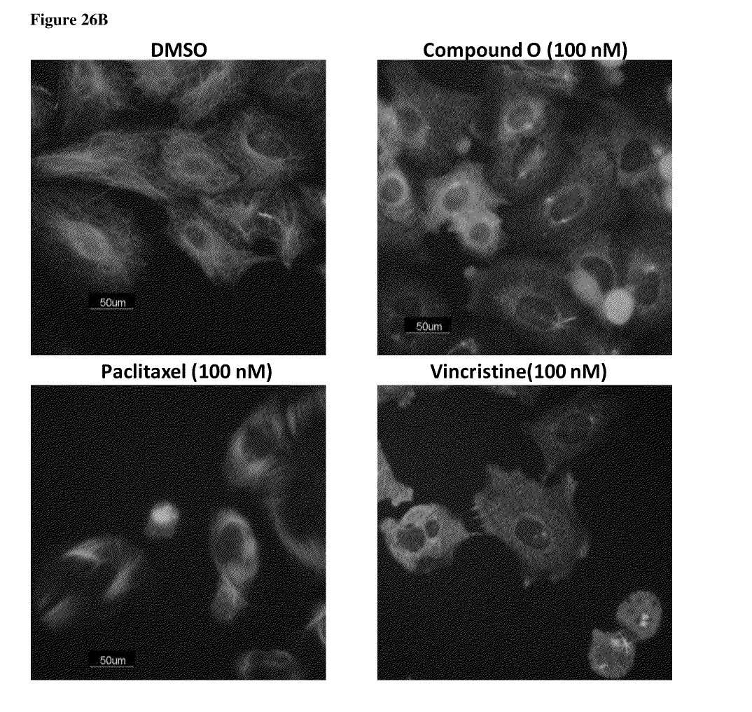

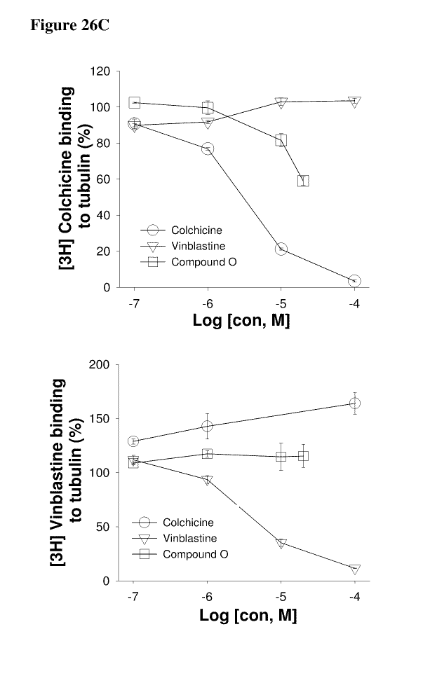

FIG. 26A shows the effect of COMPOUND O on microtubule assembly in vitro using MAP-rich tubulin. In the negative control sample, absorbance at 405 nM (A₄₀₅) increased with time. The increase of A₄₀₅ reaches a plateau in 10 minute. In the presence of 5 μM vincristine, tubulin polymerization was inhibited more than 50% compared with that of the control sample. In the presence of COMPOUND O, tubulin polymerization was inhibited in a concentration-dependent manner COMPOUND O at 0.04 μM showed no inhibition which is similar to that of negative control. COMPOUND O at 5 μM showed similar inhibition pattern to that of the positive control (vincristine at 5 μM). In the presence of 5 μM paclitaxel (a tubulin polymerization enhancer), tubulin polymerization was further enhanced compared to that of the negative control.

FIG. 26B demonstrates the inhibition of microtubule organization in A549 cells by 20 hr treatment of COMPOUND O. The microtubule network in control cells exhibited normal organization and arrangement (FIG. 24B DMSO). In contrast, paclitaxel treatment resulted in microtubule polymerization with an increasing in the density of cellular microtubules (FIG. 24B Paclitaxel). Furthermore, COMPOUND O treatment resulted in findings similar to those of vincristine-induced microtubule changes (FIG. 24B COMPOUND O & vincristine).

FIG. 26C demonstrates that COMPOUND O interacts with tubulin via a colchicine-binding site using a spin column assay. As shown in FIG. 26C (upper panel), tubulin incubation with [³H]colchicine in the presence of unlabeled colchicine reduced the amount of [³H]colchicine found in the flow-through in a concentration-dependent manner. Similarly, COMPOUND O reduced the amount of [³H]colchicine in the flow-through in a concentration-dependent manner. As a negative control, vinblastine did not influence the amount of [³H]colchicine in the flow-through. We also examined whether COMPOUND O interact with tubulin by binding to a vinblastine-binding site on tubulin using spin column assay. As shown in FIG. 26C (lower panel), tubulin incubation with [³H]vinblastine in the presence of unlabeled vinblastine reduced the amount of [³H]vinblastine found in the flow-through in a concentration-dependent manner. By contrast, neither colchicine nor COMPOUND O affected the amount of [³H]vinblastine in the flow-through.

### Example 27

### COMPOUND O Induces Apoptosis in Cancer Cells

To test if COMPOUND O compound induces apoptosis in cancer cells, the A549 human lung cancer cells were treated with 37 nM COMPOUND O and 37 nM paclitaxel. Cell apoptosis and death detection was measured by Cell Death Detection ELISA kit (Roche Applied Sciences), according to the assay protocol from the kit. Briefly, 5000 cells were seeded into each well of the 96-well plate. After 24 hours incubation, the cells were treated with 37 nM of COMPOUND O and paclitaxel, a concentration close to the IC50 value of the cell proliferation. Cells were harvested and lysed 24 hr, 48 hr or 72 hr post drug treatment. Twenty microliters of lysate were removed and transferred to streptavidin-coated microplate and then incubated with anti-histone-biotin and anti-DNA-POD antibodies for 2 hr followed by adding 2,2′-azinobis-3-ethylbenzthiazoline-sulphonic-acid substrate for color development. The plate was measured at an absorbance of 405 and 490 nm in a Beckman Multimode DTX880 plate reader.

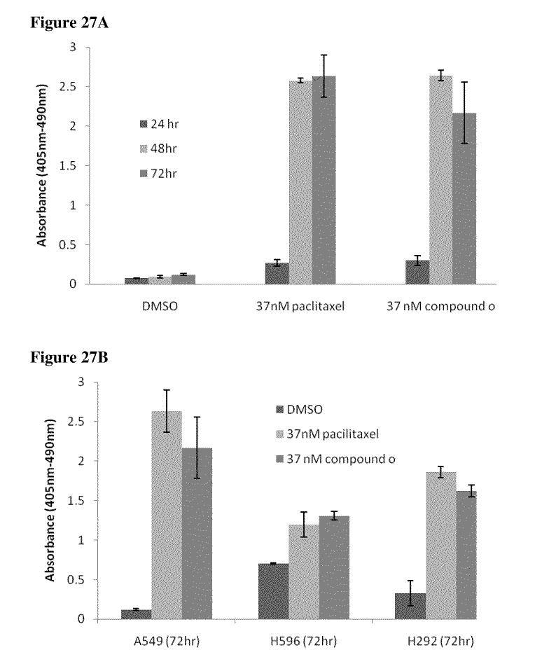

As shown in FIG. 27A, the cells treated with 37 nM COMPOUND O and 37 nM paclitaxel show strong apoptotic signals (starting from 48 hr post drug treatment), while the control cells which were only treated with DMSO showed minimal signals. This indicates that COMPOUND O induced time-dependent apoptosis in A549 lung cancer cells. In addition, we also tested two other cancer cell lines, H596 and H292. FIG. 27B shows that 37 nM COMPOUND O triggered similar level of apoptosis as 37 nM paclitaxel in these two cell lines. Taken together, COMPOUND O induced apoptosis in cancer cells.

### Example 28

### COMPOUND O Induces G2/M Cell-Cycle Arrest in Cancer Cells

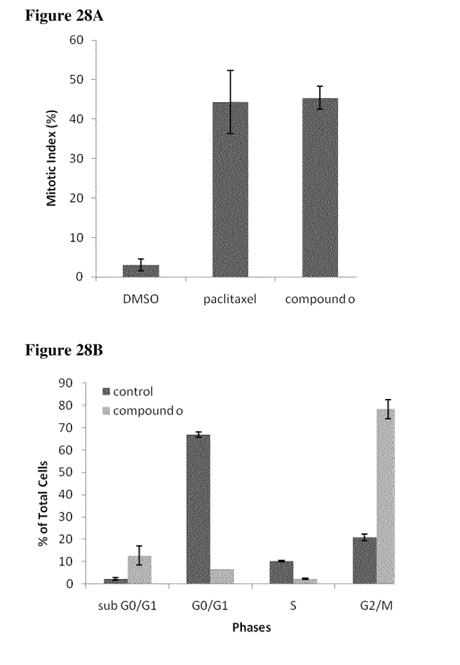

Microtubules are extremely important in the process of mitosis, during which the duplicated chromosomes of a cell are separated into two identical sets before cleavage of the cell into two daughter cells. Compounds which target microtubules such as paclitaxel and vinblastine suppress the microtubule dynamics and block the process of mitosis. As consequence, cells will be arrested at G2/M phase. To test if COMPOUND O influences the process of mitosis in cancer cell dividing, A549 human lung cancer cells were treated with 33 nM of COMPOUND O and paclitaxel, or 0.2% DMSO that served as negative control. The treated cells were stained with an antibody against phosphohistone H3, a marker for cells undergoing mitosis and mitotic arrest. FIG. 28A shows the extent of mitotic arrest by paclitaxel and COMPOUND O was quantified by the mitotic index. It was 44±8%, 45±3% and 3±1% for paclitaxel, COMPOUND O and DMSO treated A549, respectively.

In addition, we have also tested the effect of COMPOUND O on cell-cycle using flow Cytometry. Briefly, A549 cells were seeded at a density of 400,000 cells/well in 6-well tissue culture plates. Approximately 24 hours later the cells were treated with 37 nM COMPOUND O and allowed to further incubate for 24 hours. The cells were washed in PBS, trypsinized, counted and fixed in ice-cold 70% methanol and stored at 4° C. The cells were washed with PBS, stained with propidium iodide and kept on ice until flow Cytometry analysis. As shown in FIG. 28B, the cell population at G2/M phase increased significantly in cells treated with COMPOUND O compared to the cells treated with DMSO only.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of formula (I) or Formula (II): 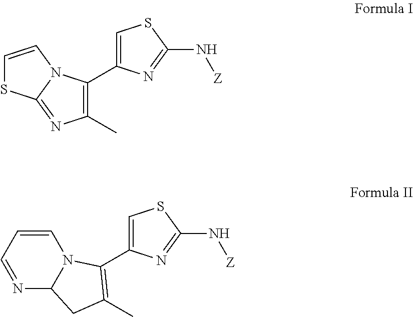
wherein Z is selected but not limited from the following substituted phenyl or heterocyclic rings: 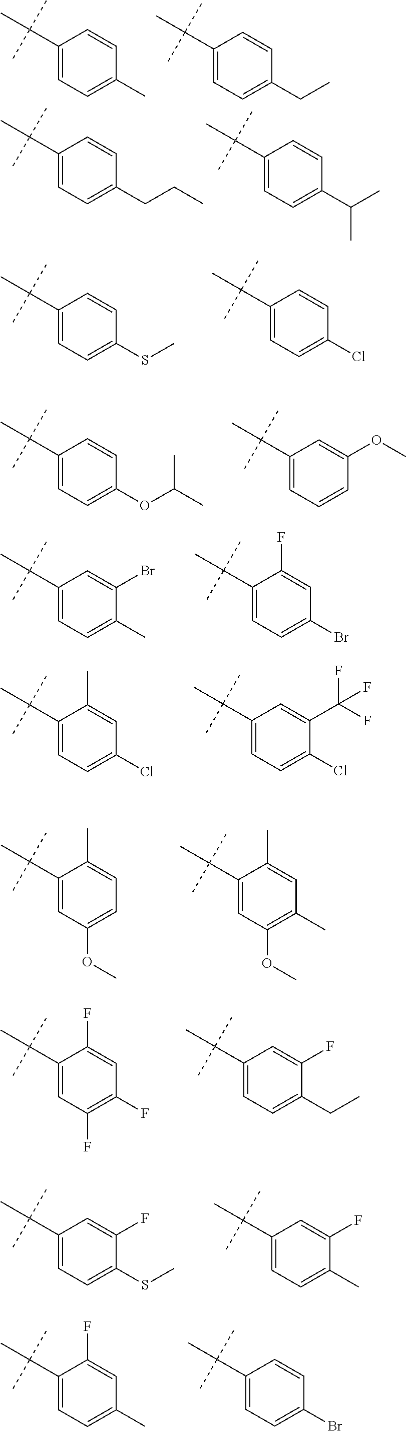 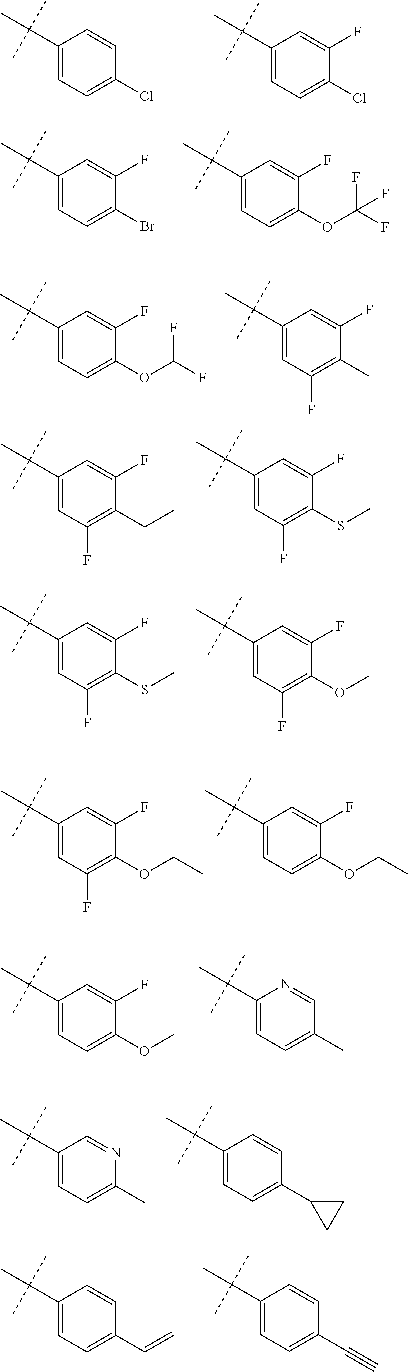 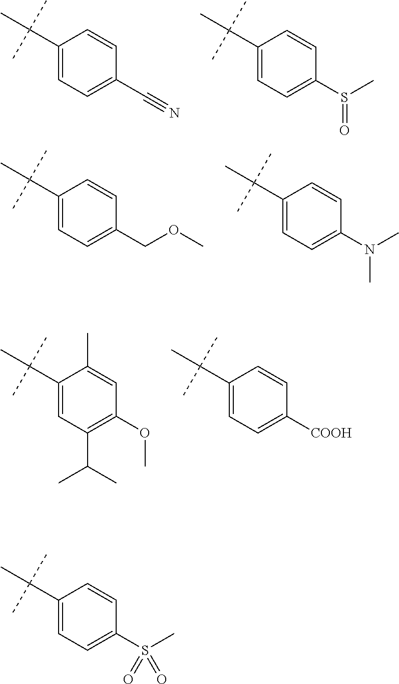
or a pharmaceutically acceptable salt thereof.

2. The compound of claim 1, which is a compound of the formula (I).

3. The compound of claim 1, which is a compound of the formula (II).

4. A compound of the formula (I) selected from

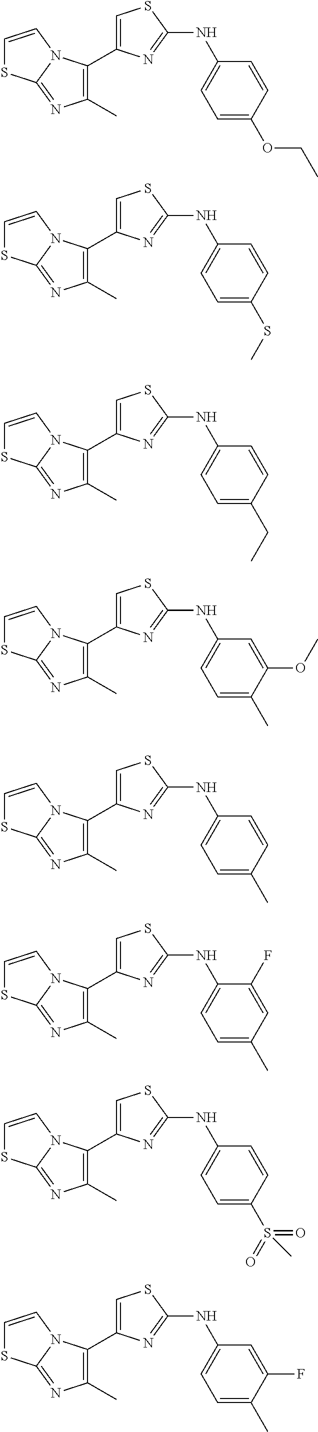

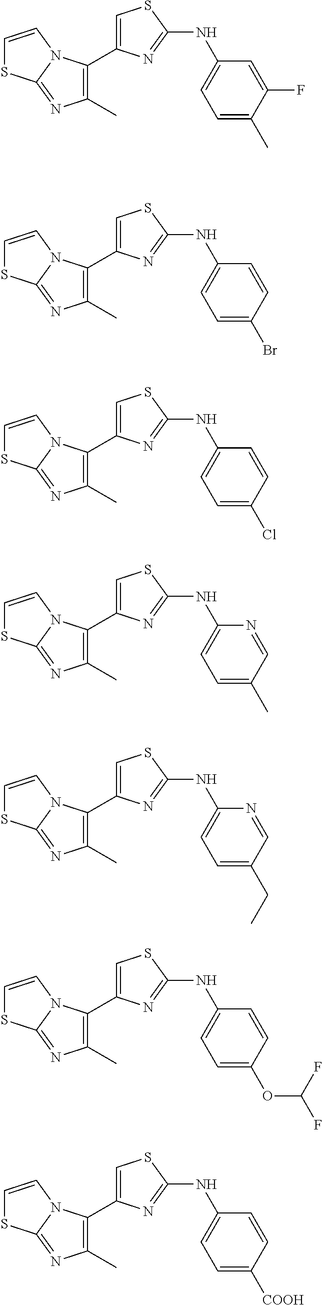

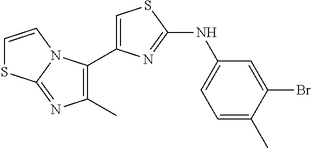

5. A compound of the formula (II) selected from

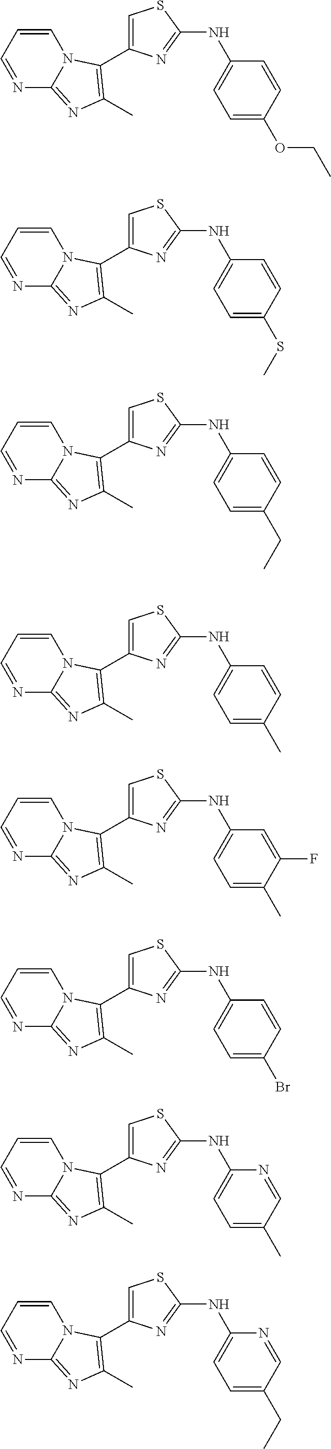

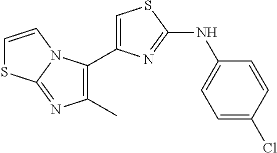

6. A pharmaceutical composition comprising a compound of claim 1 and a pharmaceutically acceptable excipient or carrier.

7. The pharmaceutical composition of claim 6, which further comprises an additional active agent.

8. The pharmaceutical composition of claim 7, wherein the additional active agent is a large molecule or a small molecule.

9. The pharmaceutical composition of claim 8, wherein the large molecule is hematopoietic growth factors, cytokines, and monoclonal or polyclonal antibodies.

10. The pharmaceutical composition of claim 8, wherein the small molecule is anti-cancer agents, antibiotics, immunosuppressive agents or steroids.

11. A method of treating, preventing or managing cancer comprising administering to a subject in need of such treatment an effective amount of a compound of claim 1.

12. The method of claim 11, wherein the subject is a human.

13. The method of claim 11, wherein the cancer is sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head cancer, neck cancer, or pancreatic cancer.

14. A method of treating, preventing or managing cancer comprising administering to a subject in need of such treatment an effective amount of a pharmaceutical composition of claim 6.

15. The method of claim of claim 14, wherein the subject is a human.

16. The method of claim 14, wherein the cancer is sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head cancer, neck cancer, or pancreatic cancer.

17. A method of treating, preventing or managing cancer comprising administering to a subject in need of such treatment an effective amount of a pharmaceutical composition of claim 7.

18. The method of claim of claim 17, wherein the subject is a human.

19. The method of claim 17, wherein the cancer is sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head cancer, neck cancer, or pancreatic cancer.

20. A compound of claim 1 for use to treat cancer.

21. The compound of claim 20, wherein the cancer is sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head cancer, neck cancer, or pancreatic cancer.

22. Use of a compound of claim 1 or a pharmaceutically acceptable salt thereof in the manufacture of a medicament for the treatment of cancer.

23. The compound of claim 22, wherein the cancer is sarcoma, epidermoid cancer, fibrosarcoma, cervical cancer, gastric carcinoma, skin cancer, leukemia, lymphoma, lung cancer, non-small cell lung cancer, colon cancer, CNS cancer, melanoma, ovarian cancer, renal cancer, prostate cancer, breast cancer, liver cancer, head cancer, neck cancer, or pancreatic cancer.
